(19) United States
(12) Patent Application Publication
(10) Pub. No.: US 2005/0232925 A1
(43) Pub. Date: Oct. 20, 2005

(54) Protease activity of thrombin inhibits angiogenesis

(76) Inventors: Vikas P. Sukhatme, Newton, MA (US); Jaime Merchan, Rochester, MN (US); Chan Barden, Allston, MA (US)

Correspondence Address: CLARK & ELBING LLP, 101 FEDERAL STREET, BOSTON, MA, 02110, US

(21) Appl. No.: 10/508,317

(22) PCT Filed: Mar. 14, 2003
(86) PCT No.: PCT/US03/08121

§ 371 Date: Jun. 16, 2005

Related U.S. Application Data
(60) Provisional application No. 60/365,165, filed on Mar. 18, 2002.

(51) Int. Cl. A61K 39/395; A61K 38/10; A61K 38/08; A61K 31/401

(52) U.S. Cl. 424/155.1; 514/14; 514/16; 514/423

(57) ABSTRACT

The present invention features pharmaceutical compositions and methods to inhibit angiogenesis, with implications to cancer therapy. These methods are based on the discovery that activated thrombin has antiangiogenic activity and that this antiangiogenic activity is at least in part, mediated through the activation of a class of thrombin receptors termed, Protease Activated Receptor (PAR). Pharmaceutical compositions and methods are also directed to a class of proteases which mediate this activation, particularly the urokinase plasminogen activator (uPA) polypeptide.

### FIELD OF THE INVENTION

The invention relates to the use of thrombin and its effectors to inhibit neovascularization.

### BACKGROUND OF THE INVENTION

Angiogenesis is the growth of new blood vessels. Angiogenesis occurs naturally in mammals for healing wounds and for restoring blood flow to tissues after injury or insult. In females, angiogenesis also occurs during the monthly reproductive cycle (to rebuild the uterus lining, to mature the egg during ovulation) and during pregnancy, to build the placenta. The process of angiogenesis is, in part, governed by angiogenesis-stimulating factors and negatively regulated by angiogenesis inhibitors. When angiogenic factors are produced in excess of angiogenesis inhibitors, neovascularization is favored. Conversely, when inhibitors are present in excess of stimulators, angiogenesis is stopped.

Angiogenesis-associated diseases include, but are not limited to cancer, including HIV Kaposi's sarcoma, rheumatoid arthritis, psoriasis, pyogenic granuloma, diabetic retinopathy, macular degeneration, corneal graft neovascularization, hypertrophic scarring, angiofibroma, Osler-Weber syndrome, neovascular glaucoma, and scleroderma.

Considerable data point to the importance of angiogenesis both in primary tumor growth as well as for metastases. Tumor angiogenesis is a complex process that is controlled by a balance between angiogenesis activators and inlubitors. Proangiogenic molecules include vascular endothelial growth factor, interleukin 8 (IL-8), and basic fibroblast growth factor (bFGF), among others. Angiogenesis inhibitors can be divided into several classes. One group consists of antibodies to proangiogenic factors or their receptors, as well as small molecule inhibitors of signaling pathways triggered by these agents. A second class consists of endogenous proteins, such as thrombospondin and platelet factor-4. A third group, which has attracted considerable attention recently, includes fragments of endogenous proteins where the parent protein is devoid of antiangiogenic activity or is even proangiogenic. Examples include fragments of the extracellular matrix (endostatin, restin, tumstatin, and canstatin), as well as fragments or conformational states of molecules involved in coagulation and fibrinolysis (angiostatin: the first four kringle domains of plasminogen and the antiangiogenic conformation of anti-thrombin III). The antiangiogenic and anti-tumor activities of these molecules have also been demonstrated in vivo. Though some of these antiangiogenic protein fragments are in clinical trials, they are difficult to produce and are not orally available.

### SUMMARY OF THE INVENTION

The present invention features methods to inhibit angiogenesis, which may be used, for example, in cancer therapies. These methods are based on the discovery that activated thrombin has antiangiogenic activity and that this antiangiogenic activity is at least in part, mediated through the activation of a class of thrombin receptors termed, Protease Activated Receptor (PAR).

Accordingly, in a first aspect, the invention features a method for the treatment of angiogenesis-associated diseases. The method includes the steps of administering a therapeutic amount of a pharmaceutical composition comprising a Protease-Activated Receptor (PAR) agonist capable of binding directly to the PAR receptor.

In a second aspect, the invention features another method for the treatment of angiogenesis-associated diseases. This method includes the steps of administering a therapeutic amount of a compound which results in activation of a Protease-Activated Receptor (PAR), the method, however, excludes administering either tissue plasminogen activator (tPA) polypeptide or a urokinase plasminogen activator (uPA), where the uPA is capable of binding to the human uPA receptor (uPA-R) if either the tPA or uPA is administered in combination with captopril.

In a desirable embodiment of the first and second aspect, the angiogenesis associated diseases include but are not limited to cancer, rheumatoid arthritis, psoriasis, pyogenic granuloma, HIV Kaposi's sarcoma, diabetic retinopathy, macular degeneration, corneal graft neovascularization, and hypertrophic scarring. Preferably the invention is directed to treating cancer.

In other desirable embodiments to the first and second aspect, the Protease Activated Receptors are the thrombin binding PARs, PAR-1, PAR-3, and PAR-4.

In still other desirable embodiments of the first and second aspect, the PAR receptors can be activated directly with polypeptide ligands to the PARs (e.g., SFLLRNPNDKYEPF, SFLLRN, SALLRN, GYPGKF, and SLIGKV) or by monoclonal antibodies. Desirably, the monoclonal antibody is modulating, more desirably, the monoclonal antibody prevents receptor internalization.

In yet still another desirable embodiment of the first and second aspect, treatment may be administered in combination with an ACE inhibitor, preferably from the group consisting but not limited to captopril, enalapril, lisinopril, benazepril, fosinopril, ramipril, quinapril, perindopril, trandolapril, and moexipril.

In a third aspect, the invention features a pharmaceutical composition comprising (i) substantially pure PAR-agonist, the agonist being capable of binding directly to the PARs; and (ii) a pharmaceutically acceptable carrier.

In a fourth aspect, the invention features a pharmaceutical composition comprising (i) a therapeutic amount of a compound which results in activation of PARs, this composition, however, does not comprise either tPA polypeptide or uPA that is capable of binding to the human uPA receptor in the absence of additional active ingredients other than captopril; and (ii) a pharmaceutically acceptable carrier.

In desirable embodiments to the third and fourth aspect, the Protease Activated Receptors are the thrombin binding PARs, PAR-1, PAR-3, and PAR-4.

In still other desirable embodiments of the third and fourth aspect, compositions can include polypeptide ligands to the PARs (e.g., SFLLRNPNDKYEPF, SFLLRN, SALLRN, GYPGKF, and SLIGKV) or by monoclonal antibodies to the PARs. Desirably, the monoclonal antibody is modulating, more desirably, the monoclonal antibody prevents receptor internalization.

In a fifth aspect, the invention features a method for the treatment of angiogenesis-associated diseases, this method involves administering a therapeutic amount of a pharmaceutical composition comprising thrombin or prothrombin to a patient diagnosed with an angiogenesis associated disease.

In a desirable embodiment to the fifth aspect, treatment also includes an anti-coagulant.

Still in another desirable embodiment to the fifth aspect, treatment also includes administering an ACE inhibitor, from the group consisting but not limited to captopril, enalapril, lisinopril, benazepril, fosinopril, ramipril, quinapril, perindopril, trandolapril, and moexipril.

In a sixth aspect, the invention features a method for the treatment of angiogenesis-associated diseases, this method involves administering a pharmaceutical composition comprising a compound that modulates PAR biological activity. The treatment does not however, comprise administering either tPA polypeptide or uPA that is capable of binding to the human uPA receptor if the treatment also involves administering captopril.

In a desirable embodiment of the fifth and sixth aspect, the angiogenesis associated diseases include but are not limited to cancer, rheumatoid arthritis, psoriasis, pyogenic granuloma, HIV Kaposi's sarcoma, diabetic retinopathy, macular degeneration, corneal graft neovascularization, and hypertrophic scarring. Preferably the invention is directed to treating cancer.

In a desirable embodiment to the sixth aspect, the Protease Activated Receptors are the thrombin binding PARS, PAR-1, PAR-3, and PAR-4.

In still other desirable embodiments to the sixth aspect, the PARs can be activated directly with polypeptide ligands to the PARs (e.g., SFLLRNPNDKYEPF, SFLLRN, SALLRN, GYPGKF, and SLIGKV) or by monoclonal antibodies. Desirably, the monoclonal antibody is modulating, more desirably, the monoclonal antibody prevents receptor internalization.

In yet still another desirable embodiment of the sixth aspect, treatment may be administered in combination with an ACE inhibitor, from the group consisting but not limited to captopril, enalapril, lisinopril, benazepril, fosinopril, ramipril, quinapril, perindopril, trandolapril, and moexipril.

In a seventh aspect, the invention features a method for identifying candidate compounds that modulate PAR biological activity, the method includes the steps of: (a) contacting a Protease-Activated Receptor to a candidate compound; and (b) measuring the binding of the compound to the PARS, wherein said binding identifies the candidate compound as a compound that is useful for modulating PAR biological activity.

In a desirable embodiment to the seventh aspect, the Protease Activated Receptors used in the screen are the thrombin binding PARS, PAR-1, PAR-3, and PAR-4.

In an eighth aspect, the invention features a method for the treatment of angiogenesis-associated diseases. This method involves administering a pharmaceutical composition comprising substantially pure urokinase (uPA) polypeptide. The urokinase polypeptide is incapable of binding to the urokinase receptor, uPA-R.

In a ninth aspect, the invention features a method for the treatment of angiogenesis-associated diseases. This method involves introducing a transgene encoding a uPA polypeptide, the uPA polypeptide being incapable of binding to the uPA receptor, to a cell. The transgene is operably linked to expression control sequences, and positioned for expression.

In a desirable embodiment of the eighth and ninth aspect, the angiogenesis associated diseases include but are not limited to cancer, rheumatoid arthritis, psoriasis, pyogenic granuloma, HIV Kaposi's sarcoma, diabetic retinopathy, macular degeneration, corneal graft neovascularization, and hypertrophic scarring. Preferably the invention is directed to treating cancer.

In a desirable embodiment of the eighth and ninth aspect, treatment may be administered in combination with an ACE inhibitor, from the group consisting but not limited to captopril, enalapril, lisinopril, benazepril, fosinopril, ramipril, quinapril, perindopril, trandolapril, and moexipril.

In another desirable embodiment of the eighth and ninth aspect, the urokinase polypeptide is a mammalian urokinase that is incapable of binding to the human urokinase receptor. Desirably, the urokinase polypeptide is mouse, rat, or human in origin. More desirably, the urokinase polypeptide is from human and the urokinase polypeptide is substantially identical to the human uPA polypeptide sequence and further comprises amino acid residue substitutions in the Ω-loop. Desirably, wherein said human uPA further comprises amino acid substitutions within the Ω-loop. In another desirable embodiment, any 2, 3, 4, 5, 6, or all 7 amino acids of the Ω-loop may be substituted with another amino acid, typically a non-conservative amino acid. Most desirably, the amino acid residue substitutions are at amino acid residues 27, 29, and 30 of the sequence ²⁴tyr-²⁵phe-²⁶ser-²⁷asn-²⁸ile-²⁹his³⁰trp in human, ²⁴tyr-²⁵phe-²⁶ser-²⁷arg-²⁸ile-²⁹arg-³⁰arg in mouse, and ²⁴tyr-²⁵phe-²⁶ser-²⁷ser-²⁸ile-²⁹arg-³⁰arg in rat.

In a tenth aspect, the invention features a method for the treatment of angiogenesis-associated diseases. This method involves introducing a transgene encoding a PAR polypeptide, and this transgene is operably linked to expression control sequences, and said transgene being positioned for expression.

In a desirable embodiment of the tenth aspect, the angiogenesis associated diseases include but are not limited to cancer, rheumatoid arthritis, psoriasis, pyogenic granuloma, HIV Kaposi's sarcoma, diabetic retinopathy, macular degeneration, corneal graft neovascularization, and hypertrophic scarring. Preferably the invention is directed to treating cancer.

In other desirable embodiments to the tenth aspect, the Protease Activated Receptors to be expressed are the thrombin binding PARs, PAR-1, PAR-3, and PAR-4.

In still another desirable embodiment of the tenth aspect, treatment may be administered in combination with an ACE inhibitor, from the group consisting but not limited to captopril, enalapril, lisinopril, benazepril, fosinopril, ramipril, quinapril, perindopril, trandolapril, and moexipril.

In yet still another desirable embodiment of the ninth and tenth aspect, the transgene is operably linked to a tissue-specific expression control sequence.

In an embodiment to any of the foregoing aspects, methods further comprise administering an additional antiproliferative agent simultaneously or within 14 days of each other in amounts sufficient to inhibit the growth of the neoplasm.

In an eleventh aspect, the invention features a method for identifying antiangiogenic molecules in serum plasma. The method includes the steps of: (i) contacting said serum plasma with a tissue protease and an ACE inhibitor; (ii) depleting said plasma of angiostatin; (iii) chromatographically separating plasma fractions; and (iv) determining angiogenic potential of said fraction. The inhibition of angiogenesis in the preceding assay identifies a fraction as antiangiogenic.

In a desirable embodiment to the eleventh aspect, mammalian serum plasma is used.

In another desirable embodiment to the eleventh aspect, the tissue protease is selected from a group consisting of urokinase, tissue plasminogen activator, and streptokinase.

In still another desirable embodiment to the eleventh aspect, the ACE inhibitor is selected from a group, which includes but is not limited to captopril, enalapril, lisinopril, benazepril, fosinopril, ramipril, quinapril, perindopril, trandolapril, and moexipril.

In yet still another desirable embodiment to the eleventh aspect, the fraction having antiangiogenic activity is further purified to allow for identification.

In a twelfth aspect, the invention features a pharmaceutical composition comprising (i) a therapeutic amount of a uPA, wherein the uPA is incapable of binding to the uPA-receptor; and (ii) a pharmaceutically acceptable carrier.

In a desirable embodiment to the twelfth aspect, the uPA polypeptide is mammalian, desirably, mouse, rat, or human uPA. More desirably, the urokinase polypeptide is from human and further comprises amino acid residue substitutions in the Ω-loop.

In another desirable embodiment, any 2, 3, 4, 5, 6, or all 7 amino acids of the Ω-loop may be substituted with another amino acid, typically a non-conservative amino acid. Most desirably, the amino acid residue substitutions of are on amino acid residues 27, 29, and 30 of the sequence ²⁴tyr-²⁵phe-²⁶ser-²⁷asn-²⁸ile-²⁹his-³⁰trp in human, ²⁴tyr-²⁵phe-²⁶ser-²⁷arg-²⁸ile-²⁹arg-³⁰arg in mouse, and ²⁴tyr-²⁵phe-²⁶ser-²⁷ ser-²⁸ile-²⁹arg-³⁰arg in rat.

In another embodiment to the twelfth aspect, the pharmaceutical composition of the twelfth aspect, are used for the treatment of an angiogenesis-associated disease. Desirably, the angiogenesis-associated disease is cancer, and more desirably, the cancer is breast cancer.

In a final aspect, the invention features a method for treating angiogenesis-associated diseases. This method involves administering a pharmaceutical composition comprising substantially pure urokinase (uPA) polypeptide and a second therapeutic agent. The urokinase polypeptide is incapable of binding to the urokinase receptor, UPA-R. Desirably, the second therapeutic agent is an antiproliferative agent. Administration of uPA polypeptides of the invention and the antiproliferative agent may be given simultaneously or within 14 days of each other in amounts sufficient to inhibit the growth of the neoplasm.

In a related embodiment to any of the foregoing aspects, pharmaceutical compositions may further comprise a second therapeutic agent, desirably, the second therapeutic agent is an antiproliferative agent.

By an “antiproliferative agent” is meant a compound that, individually, inhibits the growth of a neoplasm. Antiproliferative agents of the invention include microtubule inhibitors, topoisomerase inhibitors, platins, alkylating agents, and anti-metabolites. Particular antiproliferative agents include paclitaxel, gemcitabine, doxorubicin, vinblastine, etoposide, 5-fluorouracil, carboplatin, altretamine, aminoglutethimide, amsacrine, anastrozole, azacitidine, bleomycin, busulfan, carmustine, chlorambucil, 2-chlorodeoxyadenosine, cisplatin, colchicine, cyclophosphamide, cytarabine, cytoxan, dacarbazine, dactinomycin, daunorubicin, docetaxel, estramustine phosphate, floxuridine, fludarabine, gentuzumab, hexamethylmelamine, hydroxyurea, ifosfamide, imatinib, interferon, irinotecan, lomustine, mechlorethamine, melphalen, 6-mercaptopurine, methotrexate, mitomycin, mitotane, mitoxantrone, pentostatin, procarbazine, rituximab, streptozocin, tamoxifen, temozolomide, teniposide, 6-thioguanine, topotecan, trastuzumab, vincristine, vindesine, vinorelbine, verapamil, and the uPA octamer-capped peptide, A6.

By “ACE inhibitor” is meant an angiotensin converting enzyme inhibitor. ACE inhibitors can be selected from a group comprising, but not limited to captopril, enalapril, lisinopril, benazepril, fosinopril, ramipril, quinapril, perindopril, trandolapril, and moexipril.

By “agonist” is meant a drug or other chemical that can combine with a receptor on a cell to produce a physiologic reaction typical of a naturally occurring substance.

By “assaying” is meant analyzing the effect of a treatment, be it chemical or physical, administered to whole animals or cells derived there from. The material being analyzed may be an animal, a cell, a lysate or extract derived from a cell, or a molecule derived from a cell. The analysis may be, for example, for the purpose of detecting altered gene expression, altered RNA stability, altered protein stability, altered protein levels, or altered protein biological activity. The means for analyzing may include, for example, antibody labeling, immunoprecipitation, phosphorylation assays, and methods known to those skilled in the art for detecting nucleic acids and polypeptides.

By “cancer” or “neoplasm” is meant a cell or tissue multiplying or growing in an abnormal manner. Cancer growth is uncontrolled and progressive, and occurs under conditions that would not elicit, or would cause cessation of, multiplication of normal cells.

By “candidate compound” is meant a chemical, be it naturally-occurring or artificially-derived, that is assayed for its ability to modulate an alteration in reporter gene activity or protein levels, by employing one of the assay methods described herein. Test compounds may include, for example, peptides, polypeptides, synthesized organic molecules, naturally occurring organic molecules, nucleic acid molecules, and components thereof.

By “expression control sequences” or a “promoter” is meant a nucleic acid sequence sufficient to direct transcription. Also included in the invention are those promoter elements which are sufficient to render promoter-dependent gene expression controllable for cell type-specific, tissue-specific or inducible by external signals or agents; such elements may be located in the 5′ or 3′ regions of the native gene. Desirable promoters of the invention direct transcription of a protein in an endothelial cell; such promoters include, without limitation, promoters from the following genes: flt-1, Tie-1, Tie-2, endosialin/Tem-1, endoglin, and ICAM-2. Yet another desirable promoter of the invention directs transcription of a protein in an embryonal cell.

By “modulating” is meant conferring a change, either by decrease or increase, in the level of a receptor mediated response relative to that observed in the absence of either thrombin or PAR agonist ligand or test compound interaction with the PAR receptor or of the urokinase polypeptide with the urokinase receptor. Preferably, the change in response is at least 5%, more preferably, the change in response is 20% and most preferably, the change in response level is a change of more than 50% relative to the levels observed in the absence of thrombin, PAR agonist ligand, or test compound.

By “operably linked” is meant that a nucleic acid molecule and one or more regulatory sequences (e.g., a promoter) are connected in such a way as to permit expression and/or secretion of the product (i.e., a polypeptide) of the nucleic acid molecule when the appropriate molecules (e.g., transcriptional activator proteins) are bound to the regulatory sequences.

By “pharmaceutically acceptable carrier” is meant a carrier that is physiologically acceptable to the treated mammal while retaining the therapeutic properties of the compound with which it is administered. One exemplary pharmaceutically acceptable carrier is physiological saline. Other physiologically acceptable carriers and their formulations are known to one skilled in the art and described, for example, in “Remington: The Science and Practice of Pharmacy” (20th ed., ed. A. R. Gennaro A R., 2000, Lippincott Williams & Wilkins).

By “positioned for expression” is meant that the DNA molecule is positioned adjacent to a DNA sequence, which directs transcription and translation of the sequence (i.e., facilitates the production of, e.g., PAR polypeptide).

By “Protease-Activated Receptor” or “PAR” is meant a G protein-coupled transmembrane protein receptor capable of recognizing and binding specifically to thrombin, whereby thrombin activates this receptor by cleaving an amino-terminal exodomain to unmask a new amino terminus following binding to the receptor. The new amino terminus is able to bind intramolecularly (a ‘tethered ligand’) to the body of the extracellular region of the receptor to effect transmembrane signaling. Four PARs are known in the art of which PAR-1, PAR-3, and PAR-4 are thrombin receptors. PAR-1 is the prototypic member of this family, which belongs to the 7-transmembrane receptor super-family. PAR-1 polypeptide and nucleotide sequences can be found in the NCBI database under GenBank Accession No. XM084176.

PAR biological activity is effected upon thrombin binding and processing of the receptor. Activation of the PAR by its tethered ligand allows signaling through members of the G_{12/13}, G_{q}, and Gᵢ G-protein families resulting in platelet and leukocyte recruitment and vascular permeability in the endothelium. PAR-1 is the prototype of this receptor family and has been demonstrated to be a high affinity thrombin receptor. Thrombin cleavage of human PAR-1 results in the exposure of a new amino terminus commencing with the peptide sequence SFLLRNPNDKYEPF. Synthetic peptides to the first six amino acid residues of the tethered ligand sequences of PAR receptors have been shown to function as agonists to the receptors, independent of receptor cleavage. PAR agonist ligands can be selected from a group comprising but not limited to the polypeptides, SFLLRNPNDKYEPF, SFLLRN, SALLRN, GYPGKF, and SLIGKV. By “protein” or “polypeptide” is meant any chain of amino acids, regardless of length or post-translational modification (for example, glycosylation or phosphorylation).

By “substantially identical” is meant a polypeptide or nucleic acid exhibiting at least 75%, but preferably 85%, more preferably 90%, most preferably 95%, or even 99% identity to a reference amino acid or nucleic acid sequence. For polypeptides, the length of comparison sequences will generally be at least 20 amino acids, preferably at least 30 amino acids, more preferably at least 40 amino acids, and most preferably 50 amino acids. For nucleic acids, the length of comparison sequences will generally be at least 60 nucleotides, preferably at least 90 nucleotides, and more preferably at least 120 nucleotides.

Sequence identity is typically measured using sequence analysis software with the default parameters specified therein (e.g., Sequence Analysis Software Package of the Genetics Computer Group, University of Wisconsin Biotechnology Center, 1710 University Avenue, Madison, Wis. 53705). This software program matches similar sequences by assigning degrees of homology to various substitutions, deletions, and other modifications. Conservative substitutions typically include substitutions within the following groups: glycine, alanine, valine, isoleucine, leucine; aspartic acid, glutamic acid, asparagine, glutamine; serine, threonine; lysine, arginine; and phenylalanine, tyrosine.

By “substantially pure polypeptide” is meant a polypeptide that has been separated from the components that naturally accompany it Typically, the polypeptide is substantially pure when it is at least 60%, by weight, free from the proteins and naturally occurring organic molecules with which it is naturally associated. Preferably, the polypeptide is a polypeptide that is at least 75%, more preferably at least 90%, and most preferably at least 99%, by weight, pure. A substantially pure polypeptide may be obtained, for example, by extraction from a natural source (e.g., a fibroblast) by expression of a recombinant nucleic acid encoding the polypeptide, or by chemically synthesizing the protein. Purity can be measured by any appropriate method, e.g., by column chromatography, polyacrylamide gel electrophoresis, or HPLC analysis.

A protein is substantially free of naturally associated components when it is separated from those contaminants, which accompany it in its natural state. Thus, a protein which is chemically synthesized or produced in a cellular system different from the cell from which it naturally originates will be substantially free from its naturally associated components. Accordingly, substantially pure polypeptides not only include those derived from eukaryotic organisms but also those synthesized in E. coli or other prokaryotes.

By a “therapeutic amount” is meant an amount sufficient to result in the inhibition of angiogenesis. It will be appreciated that there will be many ways known in the art to determine the therapeutic amount for a given application. For example, the pharmacological methods for dosage determination may be used in the therapeutic context.

By “transgene” is meant any piece of nucleic acid that is inserted by artifice into a cell, or an ancestor thereof, and becomes part of the genome of the animal, which develops from that cell. Such a transgene may include a gene, which is partly or entirely heterologous (i.e., foreign) to the transgenic animal, or may represent a gene homologous to an endogenous gene of the animal.

By “urokinase plasminogen activator” or “urokinase-type plasminogen activator” or “urokinase” or “uPA” is meant a serine protease, substantially identical to the nucleotide and polypeptide sequences of GenBank Accession No. NM_{—}002658 (human), NM_{—}008873 (mouse), or NM_{—}013085 (rat). Urokinase is produced as a single chain inactive (with respect to proteolytic activity) proprotein (pro-uPA). Cleavage of the pro-uPA, producing a two-chain mature uPA, precedes activation. Other biological activities of urokinase plasminogen activator include specific cleavage of plasminogen (converting it into plasmin), activation of intracellular signaling upon binding to cell surface receptors, among them, the uPA receptor (uPA-R). The polyfunctional properties of this protein are associated with its three-domain structure. The N-terminal domain shares homology to epidermal growth factor, the central region having a kringle domain, and a C-terminal proteolytic domain containing the serine protease active center. uPA is causally involved in cancer progression, particularly in invasion and metastasis. Studies have shown breast cancer patients whose primary cancer contains high levels of uPA have a significantly worse outcome than patients with low levels.

By “urokinase plasminogen activator receptor” or “uPA-R” or “CD87” is meant a glycosylphosphatidylinositol (GPI)-anchored glycoprotein, substantially identical to the nucleotide and polypeptide sequence of GenBank Accession No. XM086017. It should be appreciated that urokinase plasminogen activator polypeptide displays specificity to uPA-R. Structure-function studies have shown the amino terminal domain of uPA binds to the uPA-receptor with high affinity. Structural determination of binding has been shown to depend on amino acid residues 24 to 30 (human uPA), and termed the Ω-loop.

Interspecies cross over of ligand-receptor binding is not observed (i.e., a murine or porcine uPA does not bind to the human uPA-receptor and conversely, a human uPA does not bind to mouse uPA-receptor). In a desirable embodiment, polypeptides of the invention include alterations to human, mouse, and rat uPA at amino acid residues, 24 to 30 (the Ω-loop), specifically to the sequence ²⁴tyr-²⁵phe-²⁶ser-²⁷asn-²⁸ile-²⁹his-³⁰trp in human, ²⁴tyr-²⁵phe-²⁶ser-²⁷arg-²⁸ile-²⁹arg-³⁰arg in mouse, and ²⁴tyr-²⁵phe-²⁶ser-27 ser-²⁸ile-²⁹arg-³⁰arg in rat. We have demonstrated that a triple mutant of murine uPA incorporating the human amino acid residue substitutions at positions 27, 29, and 30 (i.e., R27N, R29H, and R30W) has been shown to ablate binding of murine uPA to the mouse UPA-R receptor.

Other features and advantages of the invention will be apparent from the following description of the desirable embodiments thereof, and from the claims.

### BRIEF DESCRIPTION OF DRAWINGS

FIGS. 1A-I are photographs of cells showing the inhibition of endothelial cell tube formation by ex vivo treatment of plasma. HUVEC cells, plus 10% untreated or treated fresh frozen plasma (FFP) in full endothelial cell medium, were plated on each of 48-well plates previously coated with matrigel and incubated overnight at 37° C. Heparin (1 U/ml) was added to all FFP samples prior to plating. A) Untreated cells in endothelial medium. B) Normal tube formation with untreated FFP (10%). C) Significant inhibition by FFP treated with rt-PA and captopril (20%). D) Treated FFP at 10%. E) Treated FFP at 1% F) rt-PA (final concentration: 1 μg/ml) and captopril in PBS (final concentration: 0.1 μM). G) rt-PA alone, 1 μg/ml (no plasma, compare to A). H) Captopril alone, 0.1 μM (no FFP, compare to A). I) Heparin alone (1 U/ml). Bar: 250 μm.

FIG. 2 is a bar graph showing a quantitative analysis of tube formation assay (from FIG. 1). A) Untreated cells in endothelial medium. B) Untreated FFP (10%). C) FFP treated with rt-PA and captopril (20%). D) Treated FFP at 10%. E) Treated FFP at 1%. F) rt-PA and captopril in PBS (no FFP). G) rt-PA alone. H) Captopril alone. I) Heparin alone (1 U/ml, compare to A).

FIGS. 3A-E are photographs of cells showing inhibition of angiogenesis, in vivo, by systemic administration of rt-PA and captopril. The matrigel plug assay was performed as explained in materials and methods. Sections of each matrigel plug were stained by H&E and examined by light microscopy. The total number of microvessels containing red blood cells from 10 high power fields were counted and averaged. A) Mice treated with both rt-PA and captopril significantly inhibited in vivo neovascularization compared to the untreated control (+bFGF and PBS), the captopril alone group and less significantly compared with the rt-PA alone group. Each column represents the mean±SE of 3 plugs/group. B-E) Representative light microscopic appearance of matrigel plugs (H&E staining and 400× magnification). B) Unstimulated (no bFGF) control showing reduced microvessels in the plug. C) Marked neovascularization is observed in the untreated stimulated group. D) Significant reduction in neovascularization in the plugs of mice treated with rt-PA and captopril. E) In contrast, plugs of mice treated with captopril alone did not show major reduction of neovascularization as compared to the combination treatment group. Arrowheads indicate microvessels.

FIG. 4 shows the inhibition of endothelial cell proliferation caused by plasma of the patient treated with rt-PA and captopril. The assay was performed as in materials and methods. Note that at 0 hour (before rt-PA infusion began) proliferation was increased compared to baseline (no plasma control). Plasma obtained at two hours into the treatment and on the following hours caused a significant decrease in endothelial cell proliferation. The inhibitory effects persisted up to 48 hours after the start of the infusion (values represent the average of triplicate experiments). *p<0.007 (0 h vs. 12 h).

FIGS. 5A-F are photographs showing the inhibition of HUVEC tube formation by the plasma of a patient treated with rt-PA and captopril. The patient received four cycles of this treatment with the dose of rt-PA increased in each cycle. Plasma obtained at 4 hrs into the infusion was used for this assay. The assay was performed as in materials and methods, except endothelial basal medium with 1% FCS was used. A) Normal tube formation induced by the patient's plasma before the treatment. B) Mild inhibition of tube formation was observed after the first rt-PA dose. The effects were more marked when higher doses of rt-PA were used. C) Second dose. D) Third dose. E) Fourth dose. F) Quantitative analysis of the above experiment (total tube length). Bar: 250 μm.

FIGS. 6A-E are photographs of the strategies used to evaluate the contribution of angiostatin on the antiangiogenic effects of treated FFP. A) Treatment of HUVEC cells with angiostatin at 10 μg/ml. B) Affinity removal of angiostatin using lysine-Sepharose did not reduce the antiangiogenic effects of treated FFP (lysine flow through). C) The lysine-bound fraction had a mild inhibitory effect on tube formation. D) Immunodepletion using a monoclonal antibody against human angiostatin did not affect the antiangiogenic activity of treated plasma. Bar: 250 μm. E) Western blot using anti-angiostatin antibody (polyclonal against kringle 1) showing increased levels of angiostatin by treated plasma and effective removal by affinity chromatography and immunoprecipitation. Lane 1. Pure human angiostatin (kringles 14). Lane 2. Untreated FFP. Lane 3. Treated FFP. Lane 4. Lysine-Sepharose flow-through. Lane 5. Immunodepleted treated FFP.

FIGS. 7A-D are photographs of cells following fractionation of treated FFP using a 3-step gradient on a Q-Sepharose column. Treated plasma was applied to the column. Three fractions were obtained (see text), and their activities were tested in the matrigel tube formation assay. A) Flow-through/300 mM NaCl wash had no antiangiogenic effects in vitro. B) 400 mM NaCl fraction had significant antiangiogenic effects. C) 1000 mM NaCl wash showed no significant inhibition on angiogenesis. Bar: 250 μm. D) Western blot using anti-human angiostatin showed that the antiangiogenic fraction of the Q Sepharose column contained minimal amount of angiostatin-related proteins. Lane 1 Pure human angiostatin (kringles 14). Lane 2. Flow through and 300 mM NaCl wash contained most of the angiostatin in the treated FFP. Lane 3. Fraction eluted at 400 mM NaCl contained minimal angiostatin. Lane 4. Fraction eluted at 1000 mM NaCl had no angiostatin.

FIGS. 8A-F are photographs of cells following immunodepletion (IP) of plasma obtained from the patient treated with rt-PA and captopril. Plasma obtained at 4 and 8 hours into the rt-PA infusion was immunodepleted using a monoclonal antibody against angiostatin. A) Pre-treatment plasma stimulated tube formation. B) Four-hour plasma before depletion inhibited tube formation. C) The effect was retained after IP. The same effects were observed using the 8-hour plasma before (D) and after (E) IP. Bar: 250 μm. F). Western blot of patient's plasma before and after immunoprecipitation (IP) demonstrating effective removal of angiostatin. Lane 1. Four-hour plasma pre IP. Lane 2. Four-hour plasma post-IP. Lane 3. Eight-hour plasma pre IP. Lane 4. Eight-hour plasma post-IP. Lane 5. Plasma before rt-PA infusion.

FIG. 9 shows a 4-15% gradient SDS-PAGE gel showing the progress of protein purification. Approximately 10-20 μg of proteins were loaded onto each lane. Proteins were visualized by Coomassie Brilliant Blue staining.

FIGS. 10A-J are photographs of cells treated with fractions derived from the purification of antiangiogenic activity in tPA/captopril-treated human plasma. (A) Negative control; (B) In vitro tPA/captopril-treated plasma; (C) HiTrap QXL flow-through fraction; (D) HiTrap QXL 400-mM NaCl fraction; (E) HiTrap QXL 1000-mM NaCl fraction; (F) HiTrap Blue flow-through fraction; (G) HiTrap Blue 1.5-M NaCl fraction; (H) HiTrap Blue 2-M guanidine hydrochloride fraction; (1) Ni-NTA flow-through fraction; (J) Ni-NTA 200-mM imidazole fraction.

FIGS. 11A-L are photographs of cells showing the effects of the protease activity of thrombin on endothelial cell tube formation in vitro. (A) Negative control; (B) 5 μg/ml prothrombin; (C) 10 μg/ml prothrombin; (D) 1 U/ml thrombin; (E) 5 U/ml thrombin; (F) 10 U/ml thrombin; (G) 10 U/ml thrombin with 200 U/ml lepirudin; (H) 10 g/ml prothrombin with 200 U/ml lepirudin; (I) 10% (v/v) tPA/captopril-treated plasma; (J) 10% (v/v) tPA/captopril-treated plasma with 200 U/ml lepirudin; (K) 10% (v/v) untreated plasma with 200 U/ml lepirudin; (L) 200 U/ml lepirudin.

FIGS. 12A-D are photographs of cells showing the effects of thrombin-receptor-activating peptide (TRAP) on endothelial cell tube formation in vitro. (A) Negative control; (B) 1 μM; (C) 10 μM; (D) 100 μM of TRAP respectively.

FIGS. 13A-E are photographs showing Matrigel-plug assays to evaluate angiogenesis in vivo. (A-D): H&E stained sections of matrigel plugs excised from mice 10 days post implantation. (A) No bFGF; (B) 250 ng/ml bFGF; (C) 250 ng/ml bFGF and 10 ρg/ml prothrombin; (D) 250 ng/ml bFGF and 250 μM TRAP. Arrowheads point to red-blood-cell containing micro-vessels. (E) The results of the matrigel-plug assays depicted in a histogram.

FIGS. 14A-F are photographs of cells showing the effects of activation of various PARs on endothelial cell tube formation in vitro. (A) Negative control; (B) 50 μM SFLLRN; (C) 100 μM SFLLRN; (D) 100 μM SALLRN; (E) 100 μM SLIGKV; (F) 100 μM GYPGKF.

FIG. 15 is a bar graph showing the effects of tissue protease overexpression on tumor cell growth. Metastatic breast cancer cell lines (4T1) stably expressing tPA (tpa), wildtype uPa (upa), or a catalytically active, receptor binding mutant of uPA (uPA R27N, R29H, and R30W; upa mut) were implanted into BALB/c mice. Tumor growth was measured every other day for 34 days. The data collected is displayed as a line graph. Results for tumor growth were measured as a function of time. N=12 in each group of mice; EV=empty vector.

### DETAILED DESCRIPTION

We have observed that plasma serum treated with an ACE inhibitor and a protease, unlocks an angiogenic potential that can be exploited for the treatment of angiogenic disorders. We have discovered one of the factors necessary for clotting, Factor-II (widely known as thrombin), possesses this antiangiogenic potential.

The antiangiogenic potential of thrombin is at least in part mediated through the molecular interactions of thrombin to its receptor, PAR-1, a G-protein coupled receptor. Binding of thrombin to its receptor leads to the cleavage of an amino terminus of the receptor and consequently exposing a polypeptide sequence capable of intramolecularly associating with itself. Soluble versions of this ‘tethered ligand,’ upon binding to its receptor, also effect antiangiogenic activity in human endothelial cells.

Thus by introducing thrombin or ligands and mimetics that activate the thrombin receptor in regions of the body affected by angiogenic associated diseases, we can modulate the mechanisms involved in local neovascularization.

### EXAMPLE 1

### In Vitro and In Vivo Induction of Antiangiogenic Activity by Plasminogen Activators and Captopril

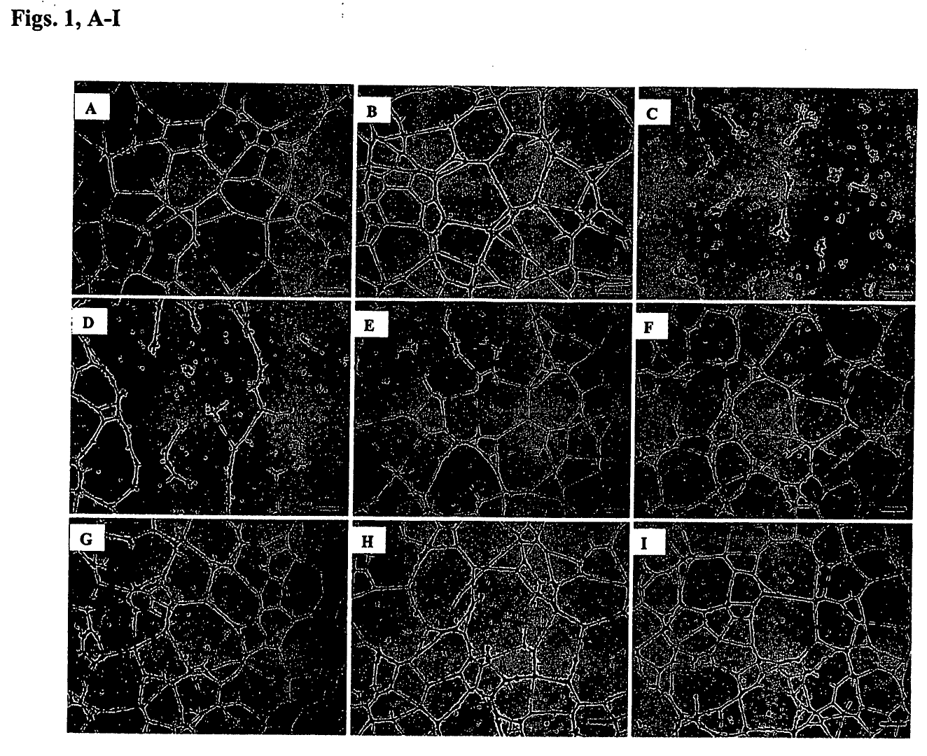

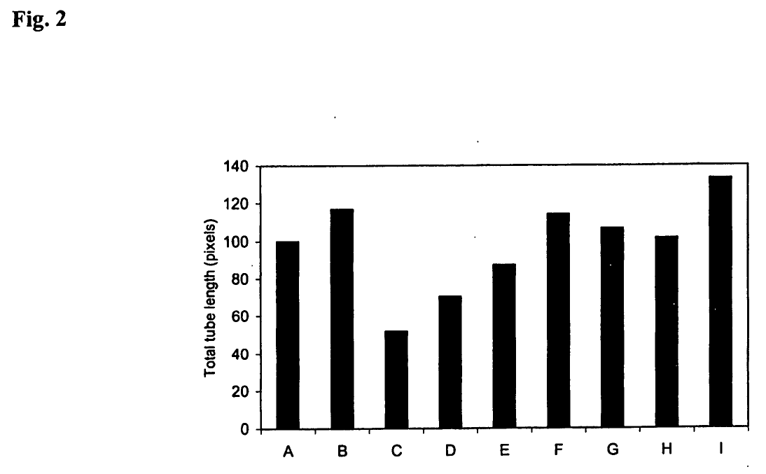

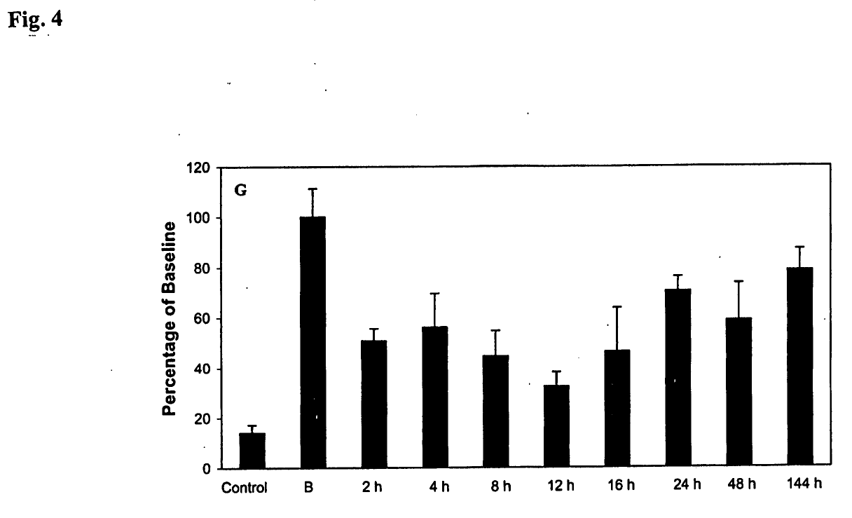

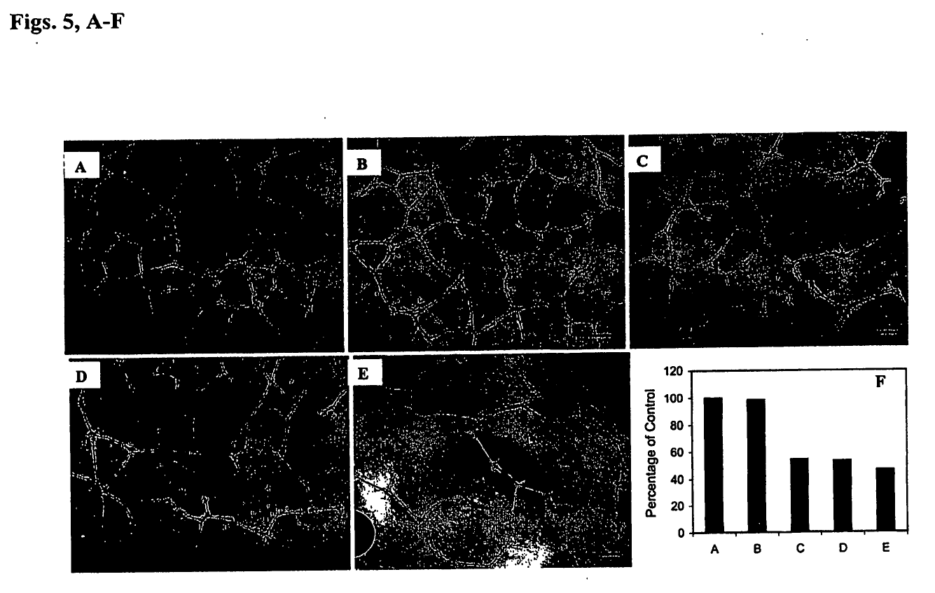

In vitro exposure of human fresh frozen plasma to rt-PA and captopril induced significant in vitro antiangiogenic activity as assessed by the matrigel tube formation assay (FIG. 1 and FIG. 2). Pharmacokinetic studies have shown that plasma concentrations of 0.1 to 1 μM are achieved by doses of captopril of 25 to 37.5 mg three times a day. Plasma concentrations of tissue plasminogen activator in healthy volunteers and patients treated for myocardial infarction are in the range of 0.5 to 1.8 μg/ml receiving doses of 0.004 mg/kg/min. The concentrations used in our assays are within this range. The in vitro findings were extended to the in vivo setting by treatment of a patient with metastatic sarcoma with captopril and low dose rt-PA. A potent antiangiogenic effect was induced in her plasma (FIGS. 4 and 5). The demonstration that the treated patient's plasma inhibited endothelial cell proliferation and capillary tube formation suggests that biologically relevant antiangiogenic effects can be induced at clinically tolerable doses of rt-PA and captopril. Moreover, the finding that systemic administration of rt-PA and captopril into mice decreased neovascularization in the matrigel plug assay is further indication that the effects induced by the treatment may have important biological in vivo relevance.

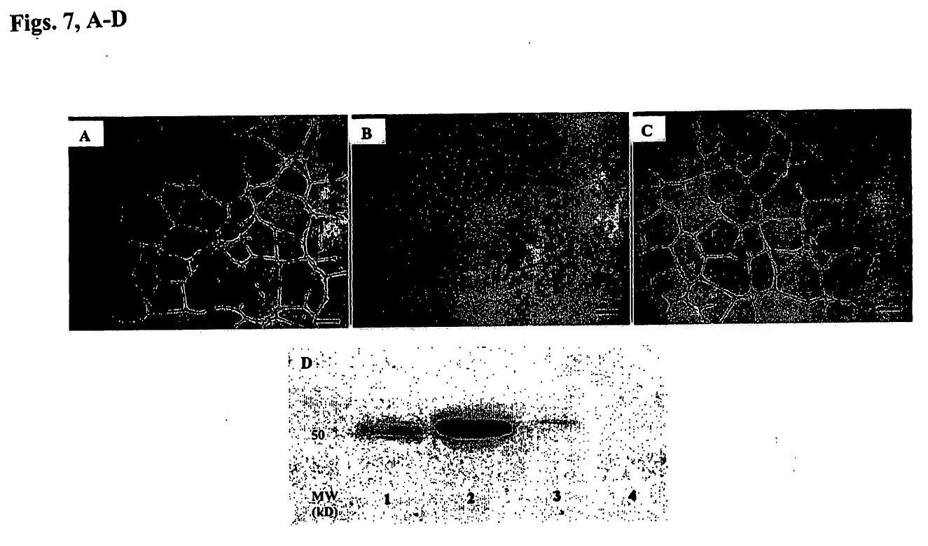

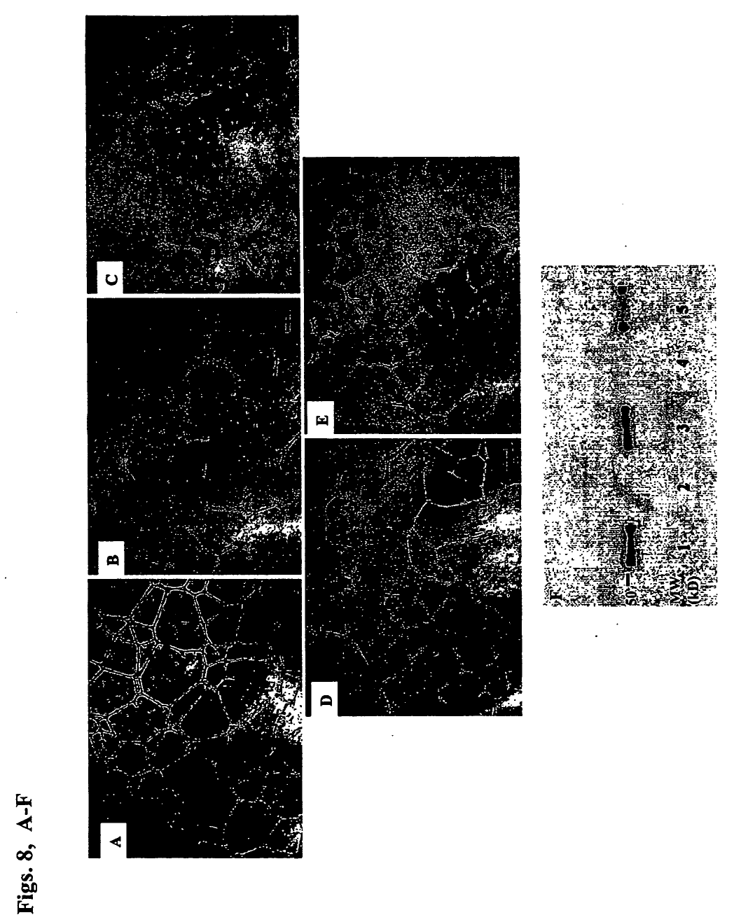

A novel finding was that the observed antiangiogenic effect from FFP exposed to rt-PA and captopril was not primarily due to the generation of angiostatin. Several lines of evidence speak to this. First, pure angiostatin at concentrations of 10 μg/ml and 50 μg/ml did not significantly inhibit tube formation (FIG. 7A and FIG. 7B). Second, affinity removal of angiostatin from FFP exposed to rt-PA and captopril did not remove the antiangiogenic activities (FIG. 7C). Third, treated FFP retained the tube formation inhibitory activities after angiostatin immunodepletion (FIG. 7D). Finally, fractionation of treated plasma demonstrated that the antiangiogenic fraction contained little or no angiostatin and the fraction that contained angiostatin had no significant inhibitory activity on in vitro angiogenesis (FIG. 8).

That angiostatin did not play a major role in the antiangiogenic effects of the treated plasma was unexpected. The data provided in this study suggest that other antiangiogenic molecules are generated as a result of the rt-PA/captopril treatment of plasma. These appeared to be separable from and more potent than angiostatin. That angiostatin did not have a significant inhibitory effect on tube formation may be related to the conditions of the assays employed. In our assays (for ex vivo treatment of plasma), we resuspended the cells in full endothelial growth media, with growth factors such as VEGF, bFGF, hEGF, IGF-1, etc. Reports that demonstrated inhibition of tube formation by angiostatin employed a less rich medium of either VEGF alone, bFGF alone, or low serum concentrations.

Another interesting finding is the duration of the antiangiogenic effects in the plasma of the patient after treatment. rt-PA is rapidly cleared from plasma, however, the endothelial antiproliferative effects of the patient's plasma persisted up to 36 hours after the infusion was stopped (FIG. 4). This evidence suggests that the effects noted were not mediated directly by rt-PA but newly generated molecule(s) with a relatively long half-life.

Role of ACE inhibitors. The results from our in vivo assays suggest that there may be a small but significant contribution of captopril to the antiangiogenic effects of rt-PA treatment (compare the matrigel plug microvessel counts from mice treated with rt-PA and captopril vs. the counts of mice treated with rt-PA alone). There are several plausible explanations. In addition to stimulating generation of angiostatin, captopril may have antiangiogenic effects by itself that could be additive or synergistic to the effects of rt-PA. Several studies have reported the antiangiogenic properties of captopril. However, the concentrations of captopril used for in vitro inhibition of angiogenesis were in the millimolar range. The antiangiogenic effects of captopril could be due to its ability to regulate extracellular (EC) tPA and PAI-1 production. There is evidence that the angiotensin-converting enzyme (ACE) plays an important role in regulating the fibrinolytic cascade by virtue of its endothelial localization and its roles in activating angiotensin and degrading bradykinin. Bradykinin is one of the most potent stimuli regulating the synthesis and secretion of tPA, and angiotensin appears to be an important regulator of PAI-1 production. Inhibiting EC ACE would theoretically down-regulate expression of PAI-1 and up-regulate expression of tpA. Captopril down-regulates expression of PAI-1 in vitro and in vivo in patients with acute myocardial infarction.

Finally, there maybe an additional component of the antiangiogenic action of rt-PA that would escape detection by our in vitro endothelial assays utilizing plasma. The generation of plasmin (from plasminogen) in the tumor microenvironment by rt-PA could trigger a series of proteolytic events leading to degradation of the tumor matrix. During the initial stages of tumor angiogenesis, fibrin formation and deposition are important to set a favorable environment for new vessel formation. rt-PA may activate fibrin bound plasminogen and enhance degradation of tumor stroma (and fibrin in particular), potentially impeding neovascularization. In view of tissue plasminogen activator's ability to promote plasmin formation when bound to plasminogen and fibrin, rt-PA administration could represent a “targeted” strategy to preferentially inhibit angiogenesis in the tumor microenvironment. This hypothesis may explain why tumors that overexpress tissue plasminogen activator are associated with less metastases in preclinical models and with a better prognosis (improved metastasis free survival and overall survival) in patients with breast cancer and melanoma.

Results

Ex Vivo Treatment of Human Plasma Inhibits in Vitro Angiogenesis. Untreated or treated fresh frozen plasma (FFP) was added to 4×10⁴ HUVEC cells to 10% (v/v) and then seeded in each of a 48-well matrigel coated plate. Untreated cells in full growth medium were used as a negative control. After 12-16 hours of incubation, treated FFP, but not untreated, exhibited a striking inhibition of EC tube formation (see images in FIG. 1, and quantitative analysis in FIG. 2). rt-PA (FIG. 1G) or captopril (FIG. 1H), alone or in combination (FIG. 1F), in the absence of plasma, did not cause significant inhibition of tube formation. The inhibitory effects decreased as the concentration of treated plasma was reduced (FIGS. 1C, 1D and 1E). Similar data was obtained with plasma from three patients with cancer exposed in vitro to rt-PA and captopril.

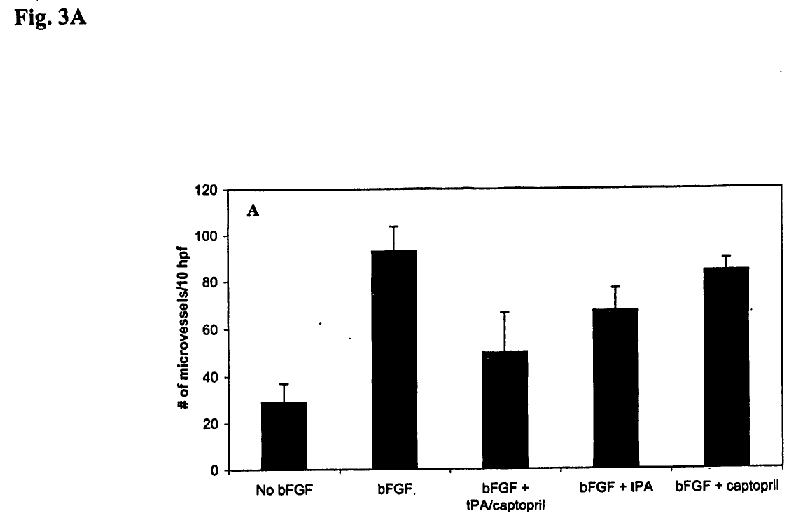

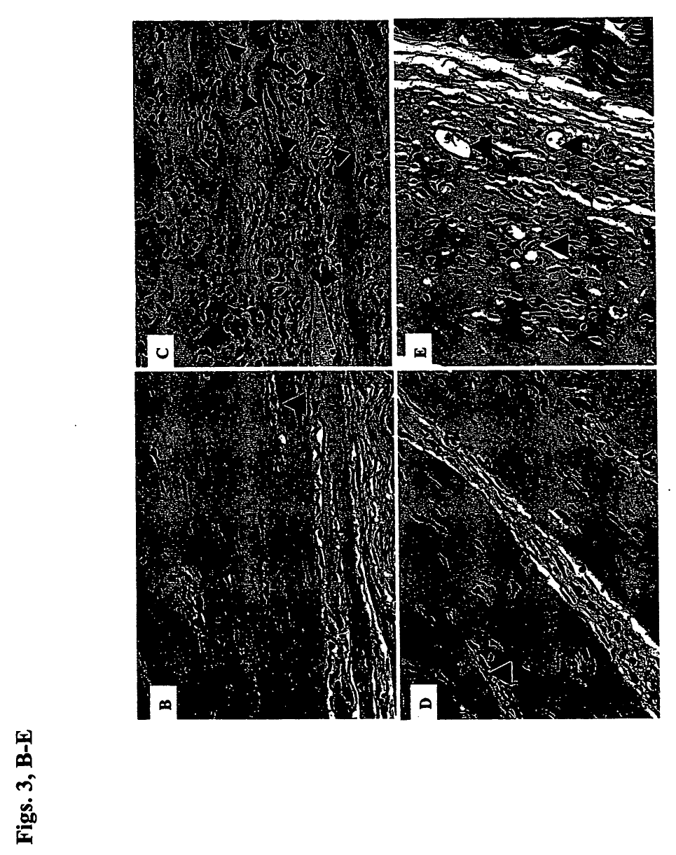

In Vivo Antiangiogenic Effects of systemic administration of rt-PA and captopril. Mice were injected with matrigel and treated as described in materials and methods. The matrigel plugs of all groups of mice (except the unstimulated control) contained bFGF as a proangiogenic stimulant. None of the treatment groups developed any significant adverse event from the treatments. At day 10, mice were sacrificed, and the plugs were analyzed Only microvessels that contained red blood cells were counted. Plugs in mice treated with the both rt-PA and captopril had significantly less microvessel counts than the groups treated with PBS, rt-PA alone, or captopril alone (FIG. 3A). Representative sections (H&E stains) of the matrigel plugs are shown in FIGS. 3B to 3E.

Induction of Antiangiogenic Effects in the Plasma of a Patient Treated with rt-PA and Captopril The patient's plasma obtained during treatment was used to perform EC proliferation and matrigel tube formation assays. At the second rt-PA dose level (0.02 mg/kg/hr), we observed a significant inhibition of HUVE cell proliferation with plasma obtained during treatment, compared to her pretreatment plasma (FIG. 4). This effect lasted for up to 48 hours after the start of the infusion and decreased significantly by 144 hours. Moreover, her plasma (during the infusion) caused a significant inhibition of HUVEC tube formation after the second dose level (FIG. 5). The antiangiogenic effect was mildly enhanced with increasing doses of rt-PA infusion (FIG. 5).

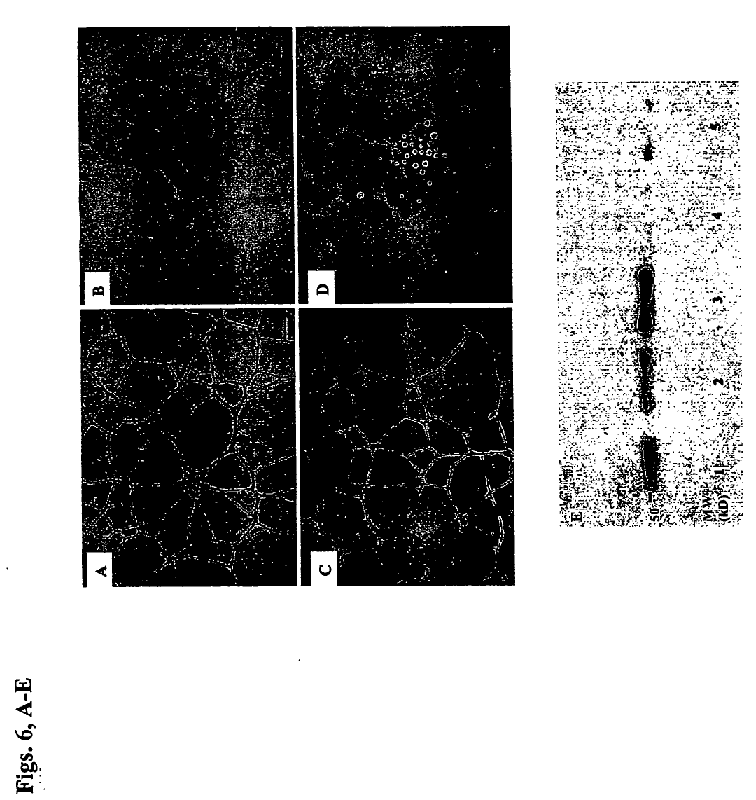

Contribution of Angiostatin to the Antiangiogenic Effects of Treated Plasma. Previous publications have demonstrated in vitro conversion of plasminogen to angiostatin after incubation of plasminogen with plasminogen activators and sulfhydryl donors. In order to determine the contribution of angiostatin to the antiangiogenic effects of treated FFP, a comparison was made between human angiostatin and treated FFP. Angiostatin at 10 μg/ml (FIG. 6A) or 50 μg/ml did not significantly inhibit tube formation in the matrigel assay. In comparison, a significant inhibitory effect was achieved with 10% treated FFP (FIG. 1B), which theoretically should contain approximately 10 μg/ml of angiostatin, assuming full conversion of plasminogen (200 μg/ml in 100% plasma) to angiostatin.

Next, affinity chromatography using lysine-Sepharose was performed on treated FFP. A western blot analysis demonstrated removal of angiostatin from the treated plasma (FIG. 6E, compare lane 4 vs. lane 3). The tube formation inhibitory effects of plasma depleted of angiostatin (flow through) were retained (FIG. 6C) and appeared similar to treated FFP before affinity removal of angiostatin. The lysine bound fraction had a very mild inhibitory effect on tube formation (FIG. 6B).

To further confirm this observation, treated plasma was immunodepleted of angiostatin using monoclonal antibodies against human angiostatin. Successful removal of angiostatin was demonstrated by western blot analysis (FIG. 6E, lane 5). The antiangiogenic effects of treated plasma, as assessed by the matrigel tube formation assay, were also retained after angiostatin immunodepletion (FIG. 6D).

Finally, we performed ion exchange chromatography using a Q-Sepharose column on the treated plasma. Three fractions were obtained (flow through after loading at 150 mM combined with a wash at 300 mM NaCl, eluate at 400 mM NaCl, and wash at 1 M NaCl). Each fraction was used to treat HUVEC cells for the matrigel tube formation assay. The antiangiogenic effect was present in the fraction eluted at 400 mM NaCl (FIG. 7B). The flow through (FIG. 7A) and the 1 M NaCl (FIG. 7C) fractions did not have any significant effect on in vitro angiogenesis. Western blot analysis of the above fractions showed that angiostatin was present on the flow through and very minimally in the 400 mM fraction (FIG. 7D). Thus, there was a clear dissociation between the presence of angiostatin and antiangiogenic activity. Antiangiogenic Effects of the Plasma from the Treated Patient were not Completely due to Angiostatin. The next logical question was whether the antiangiogenic effects seen in vivo, from the plasma of the patient treated with rt-PA and captopril, were predominantly due to angiostatin. We performed angiostatin immunodepletion as described above. Plasma obtained from two time points during the treatment was used for immunodepletion (obtained at week 2 of treatment and at 4 and 8 hours into rt-PA infusion at 0.02 mg/kg/hr). A western blot shows angiostatin to be increased compared to pre-treatment and that it was successfully removed after immunodepletion (FIG. 8F). The inhibitory effects on tube formation is preserved on the angiostatin-depleted plasma (compare FIGS. 8B with 8C and 8D with 8E).

Experimental Procedures

Reagents. rt-PA (Genentech, San Francisco, Calif.) and captopril (Sigma-Aldrich Research, St. Louis, Mo.) were diluted in sterile phosphate buffer saline and used for the bioassays. Heparin (Elkins-Sinn Inc, Richmond, Va.) or lepirudin (Aventis Pharmaceuticals, Kansas City, Mo.) was added to FFP or patient's plasma to prevent clot formation. Matrigel (Collaborative Biomedical Products, Bedford, Mass.), a basement membrane preparation from the Engelbreth-Holm-Swarm (EHS) mouse sarcoma, was used at 7 mg/ml for in vitro angiogenesis (tube formation) assays and at 10 mg/ml for in vivo (matrigel plug) assays (see below). Basic fibroblast growth factor was purchased from Peprotech; Rocky Hill, N.J. The cell proliferation reagent WST-1 (Roche; Indianapolis, Ind.) was used for proliferation assays. WST-1, a tetrazolium salt, is cleaved to formazan by mitochondrial dehydrogenases in viable cells. A murine monoclonal antibody against human angiostatin (Calbiochem; San Diego, Calif.) was used for western blotting and immunodepletion. A rabbit polyclonal antibody against mouse angiostatin (Affinity Bioreagents Inc; Golden, Colo.) that cross-reacts with human angiostatin was used for western blotting. Human angiostatin (kringles 14) was obtained from Calbiochem (San Diego, Calif.). Case Report. EB is a 46 year-old female with a history of metastatic malignant fibrous histiocytoma. She has had multiple recurrences following surgical resections (pulmonary, hepatic, and subcutaneous nodules), radiation therapy, and thalidomide treatment. She refused standard chemotherapy. The patient was screened for bleeding disorders and for brain metastases and signed an informed consent. She started taking captopril at 25 mg p.o. three times a day. One week later, she received a 12-hour intravenous infusion of rt-PA. The patient received a total of four 12-hour intravenous infusions of rt-PA during a four-week period, with increasing doses each week, starting at 0.015 mg/kg/hr, 0.02, 0.03, and 0.035 mg/kg/hr (fourth dose). Blood was taken for coagulation tests (thrombin time, fibrinogen, PT, and aPTI) and for bioassays at varying time points before, during, and following the infusion. Lepirudin (5 μg/ml) was added to the patient's plasma used for bioassays to prevent clot formation. Neither during nor after the infusions did the patient experience any significant adverse reactions.

Human Plasma. Outdated fresh frozen plasma (FFP) was obtained from Beth Israel Deaconess Medical Center's blood bank. Blood from the above-described patient with cancer was collected from a peripheral vein into citrated tubes. The blood was immediately centrifuged at 3750 rpm for 10 minutes. Both fresh frozen plasma and the patient's plasma were filter-sterilized (0.2 μm sterile filters, Millipore Corporation; Bedford, Mass.) and then stored at −20° C. for future use. Captopril (1 μM) and rt-PA (10 μg/ml) were added to 1 ml of FFP and incubated for 3 hours at 37° C. before performing the bioassays.

Cell Culture. Human umbilical vein endothelial (HUVEC) cells were obtained from Clonetics (San Diego, Calif.) and used between passages 3 and 5. They were maintained in EGM2-MV medium (BioWhittaker; Walkersville, Md.) that contains endothelial basal medium (EBM-2), supplemented with 5% fetal bovine serum, gentamicin, amphotericin B, hydrocortisone, ascorbic acid, and the following growth factors: VEGF, bFGF, hEGF, and IGF-1. Cells were grown at 37° C., in a 100% humidified incubator with 5% CO₂. Cells were grown to 80-90% confluency, harvested with trypsin, and resuspended to the cell density required for each assay.

In Vitro Angiogenesis (matrigel tube formation) Assay. Unpolymerized matrigel (7 mg/ml) was placed in the wells (100 μl/well) of a pre-chilled 48-well cell culture plate and then incubated at 37° C. for 30-45 minutes. HUVEC cells were harvested in trypsin and resuspended in EC medium (4×10⁴ in 300 μl). Cells were treated with the different agents before plated onto the matrigel-coated plates. After 12 hours of incubation, tube formation was observed through an inverted photomicroscope (Nikon; Tokyo, Japan). Microphotographs of the center of each well at low power (40×) were taken with a SPOT camera (Diagnostic Instruments Inc; Sterling Heights, Mich.) and the aid of an imaging capture software (Compix Inc Imaging Systems; Township, Pa.). The microphotographs were quantitatively analyzed (total tube length) with the Simple PCI imaging analysis software (Compix). Untreated HUVEC cells in EC medium were used as a negative control, and actinomycin D (Sigma-Aldrich Research) (7.5 μg/ml) was used as a positive (inhibitory) control.

Cell Proliferation Assay. Cells (4×10³/well, in a total volume of 100 μl) were seeded into each well of a 96-well plate and maintained in the appropriate basal medium with 1% fetal bovine serum, penicillin, and streptomycin. Cells were suspended in 1% FBS and treated with the active agents and incubated at 37° C. for 72 hours. We observed that plasma was a potent stimulant of EC proliferation, and therefore we did not use any additional stimulant of proliferation (VEGF and/or bFGF) on these assays. At the end of the specified period, WST-1 (10 μl) was added to each well and incubated at 37° C. for three hours. After incubation, absorbance at 450 nm was determined using a microplate reader (Bio-Rad; Hercules, Calif.). The experiments were performed in triplicate, and the figures presented represent the average of triplicate experiments. Statistical analysis was performed using a t-test analysis (paired two sample for means analysis).

In Vivo Angiogenesis (Matrigel Plug) Assay. The matrigel-plug assay was performed as described in Maeshima et al., (Maeshima et al., J Biol Chem 275:21340-8, 2000; Maeshima et al., J Biol Chem 276:31959-68, 2001) with modifications as follows. All animal studies were reviewed and approved by the animal care and use committee of Beth Israel Deaconess Medical Center and are in accordance with the guidelines of the department of Health and Human Services. Five to six week-old male C57/BL6 mice (The Jackson Laboratories; Bar Harbor, Me.) were injected subcutaneously at the left lower abdominal wall with 0.5 ml of unpolymerized matrigel supplemented with 500 ng/ml of basic fibroblast growth factor for the stimulated controls and treatment groups and with equivalent volume of sterile PBS for the unstimulated (no b-FGF) control group. Mice (3 per group) were treated for 10 days with: 1) rt-PA (60 μg subcutaneously/day) and captopril (150 μg intraperitoneally/day). 2) Subcutaneous rt-PA alone with equivalent volume of intraperitoneal (IP) PBS. 3) IP captopril (150 μg) with equivalent volume of subcutaneous PBS. 4) Equivalent volumes of PBS (subcutaneously and intraperitoneally). At day 10, mice were sacrificed; the matrigel plugs were removed and fixed in 4% paraformaldehyde, embedded in paraffin, sectioned, and H&E stained. Sections were examined by light microscopy, and the total number of blood vessels from 10 high power fields (400× magnification) were counted in a blinded fashion. Results shown represent the average of counts from three matrigel plugs per group.

Affinity Chromatography. Lysine-Sepharose (Pharmacia; Piscataway, N.J.) chromatography was used to separate angiostatin from the rest of the treated FFP. Briefly, a lysine-Sepharose column (6 ml) was made as per manufacturer's recommendations. The procedure was performed at 4° C. Treated plasma (10 ml) was loaded onto the column pre-equilibrated with 50 mM sodium phosphate (pH 7.5), followed by successive washes with 50 mM sodium phosphate (pH 7.5) (10 volumes), PBS (5 volumes), and 0.5 M NaCl (5 volumes). Retained proteins were eluted using epsilon aminocaproic acid (Sigma-Aldrich Research, St. Louis, Mo.) at 200 mM in water. The eluted protein was dialyzed (dialysis membrane with a molecular weight cutoff of 3000 obtained from Pierce Chemical Company; Rockford, Ill.) against PBS (4 L) for 48 hours, concentrated to the original volume of the plasma, filter sterilized, and stored at −20° C. for future use.

Angiostatin Immunoprecipitation of Plasma. Treated plasma (200 μl) was incubated overnight with a monoclonal antibody against human angiostatin (32 μg/ml) and rocked at 4° C. The next day, protein A+G agarose (50 μl) was added and rocked for 2 hours at 4° C. The samples were centrifuged (12,000 rpm for 5 minutes), and the supernatant (IP'd plasma) was stored at −20° C. for future use.

Fractionation of Treated Plasma. A series of small-scale anion exchange chromatographic steps was initially employed to optimize separation of angiostatin from the antiangiogenic activities generated in FFP by the rt-PA and captopril treatment. Treated FFP (1 ml) was exchanged into buffer A (10 mM Tris HCl pH 7.4)/50 mM NaCl by a NAP-10 column (Pharmacia). The sample was applied onto a 1-ml HiTrap QXL (Pharmacia) pre-equilibrated with buffer A/50 mM NaCl at 1 ml/min. The column was washed with the start buffer until the absorbance at 280 nm returned to baseline. Proteins were eluted by a step gradient of NaCl (50-mM increments) until 500 mM NaCl was reached. The column was then washed with buffer A/1M NaCl. All fractions were concentrated and exchanged into 1×PBS before testing for activities. The antiangiogenic activities were eluted between 300 and 400 mM NaCl fractions. Preparative-scale separation was performed by applying treated FFP onto a 20-ml HiPrep 16/10 Q XL column (Pharmacia). The column was washed extensively with Buffer A/300 mM NaCl. Absorbed proteins were eluted from the column sequentially with buffer A/400 mM NaCl and buffer A/1 M NaCl. All fractions were concentrated and exchanged into 1×PBS and stored at −20° C. for further use.

SDS PAGE and Western Blot. Protein samples diluted with SDS/DTT were separated by 4-20% polyacrylamide gel electrophoresis (pre-cast gels, Bio-Rad). This was followed by electroblotting onto a polyvinylidenedifluoride (PVDF) membrane. After blocking with 2% BSA in Tris-buffered saline/tween-20 (TTBS) for 1 hour, the PVDF membrane was incubated overnight with the polyclonal angiostatin antibody (2 μg/m). After washing with TTBS, the membrane was incubated with a horseradish peroxidase-conjugated secondary antibody (Amersham Corporation; Arlington Heights, Ill.; 1:5000 dilution) for 1 hour. The protein bands were detected using SuperSignal® West Pico Chemiluminescent Substrate (Pierce Chemical Company; Rockford, Ill.).

### EXAMPLE 2

### Protease Activity of Thrombin Inhibits Angiogenesis

We have discovered that treatment of whole human plasma with tPA in combination with captopril in vitro resulted in the generation of an antiangiogenic activity. However, the induced antiangiogenic moiety in this treated plasma was unlikely to be angiostatin, as removal of angiostatin by either immunodepletion or lysine Sepharose chromatography did not diminish the antiangiogenic activity present in the tPA/captopril-treated plasma. Here we describe a series of steps to purify and identify the protein with this novel antiangiogenic activity.

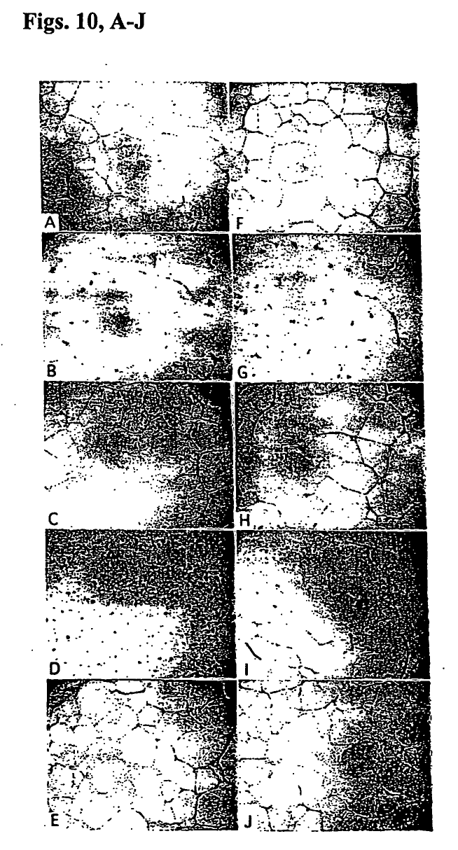

We have developed an efficient three-column scheme. First, the treated plasma is subjected to anion exchange chromatography, yielding a fraction that contains most of the antiangiogenic activity (FIG. 10D) but accounts for <2% of the input proteins. Interestingly, the potency of this fraction (and the active fractions from the later columns) is more than that of the tPA/captopril-treated plasma prior to purification (compare FIGS. 10B and 10D). We believe this difference may reflect the fact that whole plasma contains angiogenic factors that are not present in the active fractions.

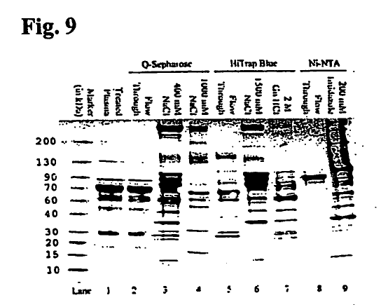

Antiangiogenic activity is further purified by Cibacron Blue F3G-A-affinity (Blue Sepharose) and Ni-NTA chromatography. The Blue Sepharose column effectively removes about 60% of the bulk proteins (FIG. 9, lane 6). Most importantly, under our conditions, the Ni-NTA column essentially absorbs all of the remaining contaminating proteins (FIG. 9, lane 8) while the active species flows through into the ‘flow-through’ fraction (FIG. 101).

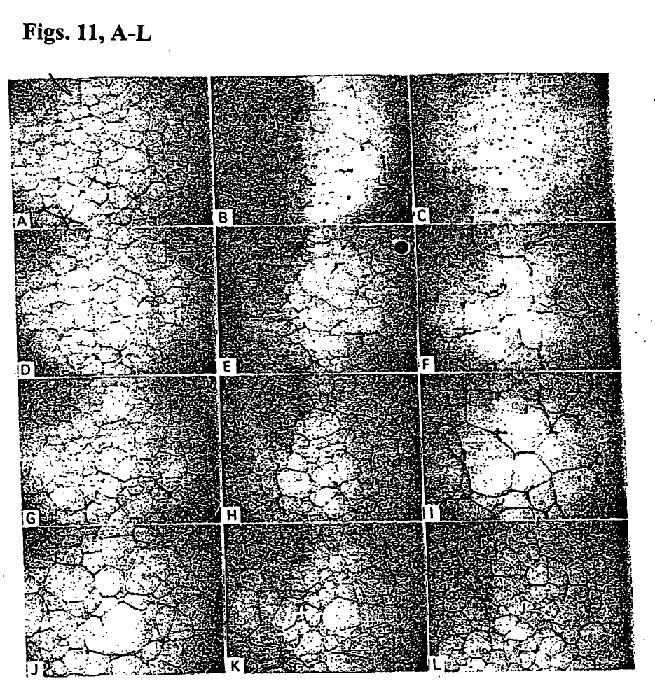

The purified antiangiogenic protein was submitted to protein sequencing by MALDI-TOF. The spectral data revealed that the antiangiogenic protein purified from in vitro tPA/captopril-treated plasma to be prothrombin with a molecular weight of 71904.7. Since it was likely that addition of tPA/captopril in plasma under these conditions initiated a cascade of proteolysis, we wanted to determine whether prothrombin was proteolytically altered during the treatment and thus became antiangiogenic. To address this possibility, a sample of commercially purchased prothrombin was submitted for analysis by mass spectroscopy and was determined to have a molecular weight of 71441, which indicated that it was actually smaller than the prothrombin purified from the tPA/captopril-treated plasma. This subtle difference in the molecular weights may reflect differences in glycosylation or extent of gamma carboxylation at the Gla domain of prothrombin. It is also possible that some protein degradation occurred in the commercially available protein. However, the minor difference in the molecular weights apparently was not related to prothrombin's antiangiogenic activity, because the commercial prothrombin was also antiangiogenic (FIGS. 11B and 11C) and appeared to have the same potency as the prothrombin purified from the tPA/captopril-treated plasma. Furthermore, we also showed that prothrombin purified from untreated human plasma using the same purification scheme was also antiangiogenic. Since prothrombin activation normally leads to clot formation, the antiangiogenic activity of thrombin in whole plasma was not previously evaluated. We showed that addition of tPA and captopril unmasked this activity in whole plasma. We hypothesize that this induction of activity may be due to the decrease in the clotting potential of the plasma post treatment. Consistent with this notion, when fibrinogen was removed by either anionic or hydrophobic interaction chromatography, the fraction containing prothrombin was potently antiangiogenic. Furthermore, fractions of untreated plasma that were devoid of prothrombin were not antiangiogenic and could not be rendered antiangiogenic by the tPA/captopril treatment.

Collectively, our data suggest that prothrombin is antiangiogenic in vitro and in vivo. Although normally suppressed in plasma, this activity of prothrombin was unmasked by treatment with tPA/captopril in vitro.

Structurally, prothrombin can be divided into four domains: a Gla domain, a kringle 1 domain, a kringle 2 domain, and a serine protease domain. The Gla and kringle 1 domains together are often referred as the fragment 1 of prothrombin, whereas the kringle 2 domain is also called fragment 2. Previously it was reported that both fragments 1 and 2 of prothrombin inhibited endothelial cell proliferation in vitro and angiogenesis in the chorioallantoic membrane of chick embryo. In this report, we examined the contribution of the protease activity of thrombin to the induced antiangiogenic activity in tPA/captopril-treated plasma. We showed that thrombin significantly inhibits endothelial cell tube formation at 10 U/ml (FIG. 11F). This effect was completely blocked by the addition of lepirudin (FIG. 11G), a specific thrombin inhibitor. Significantly, the inhibitory effect of prothrombin (FIG. 11C) was also blocked by lepirudin (FIG. 11H). Since prothrombin is devoid of proteolytic activity, our data suggest that prothrombin became proteolytically active when incubated with endothelial cells on matrigel in vitro. Western blot analyses showed that prothrombin was proteolytically cleaved into smaller fragments during the assay.

Based on our results, thrombin modulates angiogenesis. It is known that thrombin directly affects endothelial cell functions that are regulated during the angiogenic process. For example, thrombin up-regulates endothelial cell secretion of matrix metalloproteinase (MMP)-1 and -3, affects secretion of platelet-derived growth factor (PDGF), tumor growth factor (TGF)-β1, and bFGF by endothelial cells, diminishes adhesion of endothelial cells to extracellular matrix, promotes an increase in basolateral deposition, and a decrease in apical release of the extracellular matrix proteins fibronectin, laminin, and collagens I and IV, induces endothelial cell contraction and vascular permeability, up-regulates expression of VEGF receptors (KDR and flt-1), and stimulates endothelial cell migration. In addition, it has been postulated that thrombin affects angiogenesis by activating gelatinase A. Thrombin may also affect angiogenesis indirectly through platelet activation. For example, platelets release cytokines including PDGF and VEGF and the angiogenesis inhibitor endostatin upon activation by thrombin.

Based on out results, we propose that thrombin has a bimodal effect on endothelial cell functions. We have observed a switch from stimulatory to inhibitory, as the concentration of thrombin was increased on endothelial cells in culture. This biphasic property was also reported when the effects of thrombin were assessed in either in vitro endothelial cell tube formation or in an in vivo chick chorioallantoic membrane assay.

Results

Protein Purification. We sought to identity the active antiangiogenic ingredient of tPA/captopril-treated plasma by column chromatography and used endothelial cell tube formation as a read-out. The primary reason for choosing this in vitro assay is that the effects of protein fractions can be determined rapidly, usually in less than 18 hours.

A series of small-scale anion exchange chromatographic steps can be employed to optimize separation of the sought-after antiangiogenic activity generated in plasma by the tPA and captopril treatment from the bulk of the plasma proteins. We found that the activity had a fairly strong affinity to the anion exchange resins. Therefore, plasma was first exchanged into buffer A (10 mM Tris HCl pH 7.4)/300 mM NaCl and loaded onto a HiTrap QXL column. Three fractions are collected when proteins are eluted with 300 (flow through), 400, and 1000 mM NaCl. Only the 400-mM NaCl fraction (FIG. 9, lane 3) contains a potent anti-tube formation activity (FIG. 10D), whereas the flow through (FIG. 9, lane 2) and the 1000 mM NaCl wash (FIG. 9, lane 4) have little or no activity (FIGS. 10C and 10E). Although the treated plasma (FIG. 9, lane 1) contains significant anti-tube formation activity (FIG. 10B), the activity present in the 400 mM NaCl fraction appears to be more potent than the input treated plasma (FIGS. 10B and 10D).

The active fraction from the anion exchange column is further fractionated on a HiTrap Blue-Sepharose column. We found that the antiangiogenic activity could best be purified on this column by a three-step-elution method. The sample is first loaded at 200 mM NaCl, followed by an extensive wash with the loading buffer to yield a flow-through fraction (FIG. 9, lane 5) that has no effects on endothelial cell tube formation in vitro (FIG. 10F). Using this optimized loading condition, the antiangiogenic activity is completely retained on the column and can be recovered by an elution with 1.5 M NaCl (FIG. 9, lane 6 and FIG. 10G). Tightly bound material released by a 2M guanidine hydrochloride wash (FIG. 9, lane 7) has little antiangiogenic activities (FIG. 10H) as determined by our tube formation assay.

The final step of the purification scheme involves using immobilized metal affinity chromatography (IMAC). This technique is particularly useful in separating proteins of mammalian origin due to their relatively high cysteine and histidine contents. Successful employment of IMAC depends on various factors, including the metal chosen, the structure of the chelators residing on the resins, and the loading conditions. Our pilot experiments show that the Ni/NTA combination gives the highest selectivity when the input proteins are loaded in 0.8 M sodium sulfate/50 mM sodium phosphate pH 7.0. The 1.5 M NaCl fraction from the Blue-Sepharose column is first exchanged into the loading buffer by a NAP-10 column and loaded onto a Ni-NTA Superflow column. Antiangiogenic protein passes to the flow through fraction (FIG. 9 lane 8 and FIG. 10I). Proteins bound to the resins are eluted with 200 mM imidazole (FIG. 9, lane 9) and show little antiangiogenic effects (FIG. 10J).

Under these optimized conditions, the antiangiogenic moiety in the treated plasma is purified to near homogeneity (>95% pure). SDS-PAGE reveals the active moiety appears to have a molecular weight between 70 to 90 kDa (FIG. 9, lane 8). This band was excised and submitted for protein sequencing by MALDI-TOF. Comparison of the mass spectral data of the protein fragments to a database of known protein fragments identified prothrombin as the candidate.

Prothrombin is antiangiogenic. To examine whether the prothrombin purified from tPA/captopril-treated plasma was altered, a protein sample was analyzed by mass spectroscopy. The data revealed that the prothrombin purified from tPA/captopril-treated plasma had a molecular weight of 71904.7, similar to that of a commercially available sample of prothrombin (71441). This suggests that prothrombin was most likely not altered proteolytically by the tPA/captopril treatment. The minor difference in the molecular weights apparently was not related to activities, because the commercially available prothrombin also disrupted endothelial cell tube formation (FIGS. 11B and 11C) and appeared to have a similar potency as the prothrombin purified from the treated plasma. Collectively, our data suggest that prothrombin is antiangiogenic. Although normally suppressed in plasma, this activity of prothrombin is unmasked by treatment with tPA/captopril.

Protease activity of thrombin is antiangiogenic. Since prothrombin is the inactive precursor of thrombin, we carried out a series of experiments to determine whether thrombin exhibits an antiangiogenic activity similar to prothrombin. Thrombin appears to have minimal effects on endothelial cell tube formation in vitro when used at 5 U/ml (FIGS. 11D and 11E). However, thrombin at 10 U/ml significantly inhibits tube formation (FIG. 11F). The inhibition by either prothrombin or thrombin was completely blocked by the addition of lepirudin, a specific inhibitor of thrombin (FIGS. 11G and 11H). This suggests that both prothrombin and thrombin mediates the antiangiogenic behavior through the protease activity of thrombin. Most importantly, in the presence of 200 U/ml lepirudin, endothelial cells appear to form tubes with similar efficiency when incubated with either tPA/captopril-treated (FIG. 11J) or untreated plasma (FIG. 11J). Lepirudin at 200 U/ml apparently had no effects in endothelial cell tube formation (FIG. 11K). Therefore, the majority of the induced activities in plasma were due to the thrombin's proteolytic property.

Role of PARs. Thrombin signals through a class of cell surface receptor known as the protease-activated receptors (PARs). Four PARs have been identified to date. PAR-1, 2, and 3 are expressed on human endothelial cells. Amongst these three receptors, thrombin specifically activates PAR-1 and PAR-3. The manner by which thrombin activates these receptors is novel; thrombin binds and cleaves the receptor. The newly exposed N-terminus of the receptor acts as its own ligand and activates the receptor. This leads to the development of peptides corresponding to the newly exposed N-terminal amino acid sequence of the activated receptors to activate PARs specifically.

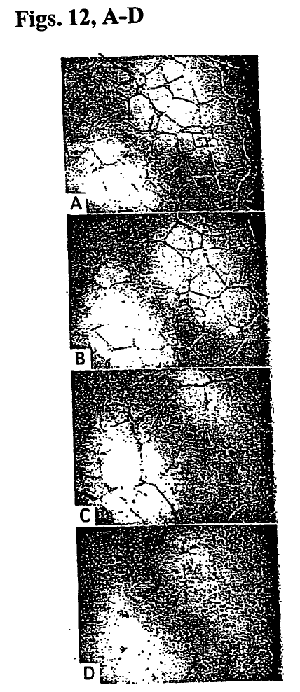

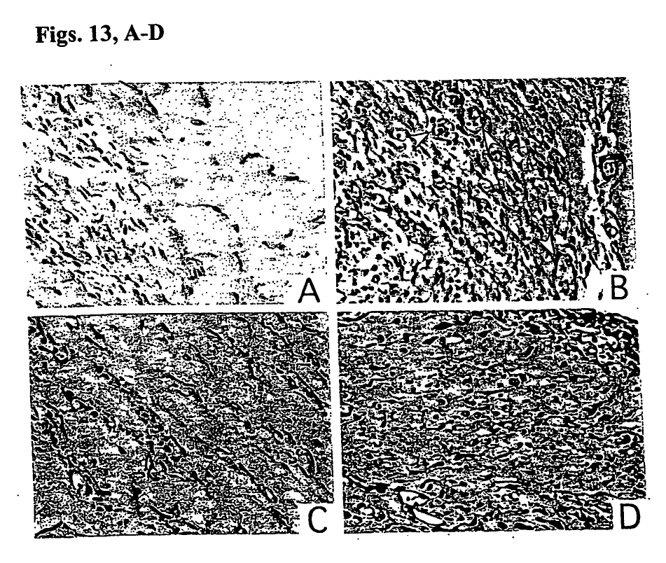

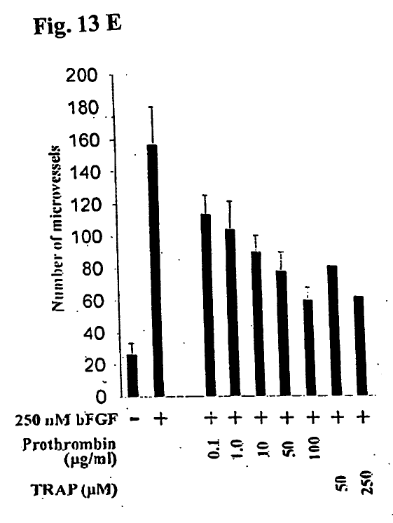

Therefore, we asked whether addition of a peptide corresponding to the N-terminal 16 amino acids of activated PAR-1 (the TRAP peptide) to endothelial cells would recapitulate thrombin's antiangiogenic effects. Indeed, TRAP dose-dependently inhibited endothelial cell tube formation in vitro (FIG. 12). This observation strongly suggests that activation of PAR-1 is sufficient to inhibit the ability of endothelial cells to form tubes in vitro. in vivo angiogenesis assays. To ascertain the in vivo relevance of this novel activity, we performed matrigel-plug assays in mice. Protirombin, TRAP, or PBS (carrier) was mixed in matrigel containing bFGF and injected subcutaneously into mice. ten days after the injections, the matrigel plugs were excised, sectioned, and stained with H&E. The number of blood vessels formed was counted and used as an indication of neovascularization. In the absence of a stimulus, minimal amount of red-blood-cell-containing micro-vessels were seen (FIG. 13A). As expected, bFGF effectively stimulated blood vessel formation in the implanted matrigel plugs (FIG. 13B). Approximately a seven-fold increase in angiogenesis was observed (FIG. 13E). However, this stimulation was significantly reduced by both prothrombin (FIG. 13C) and TRAP (FIG. 13D) in this in vivo angiogenesis model in a dose-dependent manner (FIG. 13E), consistent with the results obtained from our in vitro experiments.

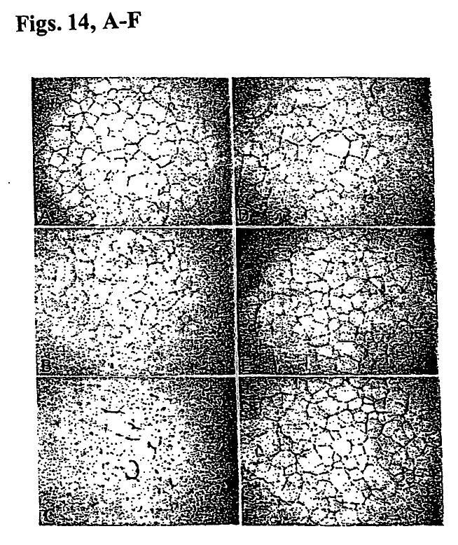

Next, we examined the effects of activating different PARs by small peptides on endothelial cell tube formation. These peptides are specific activators of either PAR-1 (SFLLRN), 2 (SLIGKV), or 4 (GYPGKF). Additionally, a mutant of the PAR-1 activating peptide (SALLRN) was also included as a control. A single amino acid mutation from phenylalanine to alanine at position 2 completely eliminated the ability of the peptide to induce platelet aggregation in vitro. Contribution of PAR-3 was not tested, because no PAR-3 activating peptide has been described so far. Activation of PAR-2 or PAR-4 on endothelial cells using the specific peptides at the concentrations tested, bore little effect on tube formation (FIGS. 14E and 14F). However, SFLLRN showed a dose-dependent inhibitory effect on endothelial cell tube formation (FIGS. 14B and 14C), consistent with the results obtained by using TRAP peptide. In contrast, the mutant peptide SALLRN showed no inhibitory effect when used at the same concentration (FIG. 14D). Collectively, these observations suggest that activation of PAR-1 inhibited HUVECs to form tubes on matrigel.

Experimental Procedures

Materials. TRAP (SFLLRNPNDKYEPF) was obtained from Sigma-Aldrich (St. Louis, Mo.). Four short peptides (SFLLRN, SALLRN, GYPGKF, and SLIGKV) were synthesized as C-terminal amides and were purified by high-pressure liquid chromatography. Prothrombin and thrombin were purchased from Calbiochem (San Diego, Calif.). Matrigel was obtained from BD Biosciences (Bedford, Mass.). Lepirudin and tPA were purchased from Aventis Pharmaceutical (Kansas City, Mo.) and Genentech (San Francisco, Calif.), respectively. bFGF was purchased from Peprotech (Rocky Hill, N.J.), and captopril was obtained from Sigma-Aldrich Research. Cells. Human umbilical vein endothelial cells (HUVECs) and EGM2-MV medium were purchased from BioWhittaker (Walkersville, Md.). Cells were cultured according to supplier's instructions.

Purification. A series of small-scale anion exchange chromatographic steps was initially employed to optimize separation of the antiangiogenic activities generated in plasma by the tPA and captopril treatment from the bulk proteins. Treated plasma (1 ml) was exchanged into buffer A (10 mM Tris HCl pH 7.4)/50 mM NaCl by a NAP-10 column (Pharmacia, Piscataway, N.J.). The sample was applied onto a 1-ml HiTrap QXL (Pharmacia) pre-equilibrated with buffer A/50 mM NaCl at 1 ml/min. The column was washed with the start buffer until the absorbance at 280 nm returned to baseline. Proteins were eluted by a step gradient of NaCl (50-mM increments) until 500 mM NaCl was reached. The column was then washed with buffer A/1 M NaCl. All fractions were concentrated and exchanged with 1×PBS before testing for activities. The antiangiogenic activities eluted between 300 and 400 mM NaCl.

We performed preparative-scale separation of protein fractions by applying treated plasma onto a 20-ml HiPrep 16/10 Q XL column (Pharmacia). The column was washed extensively with Buffer A/300 mM NaCl. Absorbed proteins were eluted from the column sequentially with buffer A/400 mM NaCl and buffer A/1 M NaCl. All fractions were concentrated and exchanged with 1×PBS and stored at −20° C. The active fraction of the Q-Sepharose column was then exchanged into 50 mM sodium phosphate pH 7.0/200 mM NaCl by NAP-10 columns and loaded onto a 5-ml HiTrap Blue Sepharose column (Pharmacia) pre-equilibrated with the loading buffer. After extensive washing, the antiangiogenic activity was eluted with 50 mM sodium phosphate pH 7.0/1.5 M NaCl. Irreversibly bound proteins were stripped off the column using 2 M guanidine hydrochloride. All fractions were concentrated and exchanged into 1×PBS and stored at −20° C. The active fraction from the HiTrap Blue column was exchanged into 0.8 M sodium sulfate/50 mM sodium phosphate by using a NAP 10 column and loaded onto a 5-ml Ni-NTA Superflow (Qiagen; Valencia, Calif.) column pre-equilibrated with the loading buffer. The column was washed extensively, following by an elution with 200 mM imidazole/50 mM sodium phosphate pH 7.0. All fractions were concentrated, exchanged into 1×PBS, and stored at −20° C.

Protein sequencing and molecular weight determination. The purified protein was subjected to protein sequencing by mass spectroscopy. Mass spectral data of the protein fragments were compared to the database NCBInr 200001111 using the search engine Mascot. This analysis revealed prothrombin as the candidate with a Mouse Score of 137. To determine molecular weights by mass spectroscopy, protein samples were further characterized using standard techniques.

In vitro treatment of human plasma. In vitro treatment of plasma with tPA and captopril may be performed. Briefly, 1 ml of plasma is incubated with 10 μg/ml tPA and 1 μM captopril at 37° C. for three hours. Samples can be stored at −20° C. until use.

Matrigel tube formation assays. Samples collected from the purification process were exchanged into 1×PBS and reconstituted into 1× concentration (by volume) compared to the input tPA/captopril-treated plasma. They were tested in the matrigel tube formation assays at 10% (v/v) as follows. Unpolymerized matrigel (7 mg/ml) was placed in the wells (100 μl/well) of a pre-chilled 48-well cell culture plate and then incubated at 37° C. for 30-45 minutes for polymerization to take place. HUVECs (4×10⁴ in 300 μl of EGM2-MV with 5% fetal bovine serum (FBS), gentamicin sulfate, amphotericin B, hydrocortisone, ascorbic acid, VEGF, bFGF, hEGF, and R³-IGF-1) were treated with the agent tested, plated onto the matrigel-coated plates, and incubated at 37° C. for 12-16 hours. Tube formation was examined (4× magnification) through an inverted phase contrast microscope (Nikon Corporation; Tokyo, Japan) and recorded by a Spot RT camera (Diagnostic Instruments Inc; Sterling Heights, Mich.) using an automated image capture software (Compix Inc Imaging Systems; Township, Pa.).

In vivo matrigel-plug assays. The matrigel-plug assay was performed as described in Maeshima et al., (Maeshima et al., J Biol Chem 275:21340-8, 2000; Maeshima et al., J Biol Chem 276:31959-68, 2001) with modifications as follows. Briefly, 500 μl of unpolymerized matrigel at a concentration of 10 mg/ml enriched with bFGF (250 ng/ml) were mixed with one of the following: PBS (vehicle), prothrombin, or TRAP. Each mixture was injected subcutaneously at the left lower abdominal wall of C57/B mice (5 to 6-week old; Jackson laboratories; Bar Harbor, Me.). At day ten, the mice were sacrificed. The matrigel plugs were then excised, fixed in 4% paraformaldehyde, embedded in paraffin, sectioned, and H&E stained. Sections were examined by light microscopy, and the total number of blood vessels from 10 high power fields (400×) were counted in a blinded fashion. Only the micro-vessels containing red blood cells were counted as positive. Results shown represent the average of counts from 4 to 5 matrigel plugs per group.

### EXAMPLE 3

### Identification of Compounds that Modulate uPA-uPA-R Biological Activity

There has been controversy in the art regarding the effects of proteases in tumor growth and progression. Based upon our results, we believe that overexpression of proteases of the plasminogen activator family (e.g., urokinase and tissue plasminogen activator) by the tumor may prevent or delay tumor growth, metastases, and improve survival.

While not wishing to be bound by a particular mechanism, we believe these proteases may degrade fibrin, known to occur in the matrix of many tumors and which is known to favor angiogenesis and support tumor cell growth. The goals of the experiments performed were to determine the effects of tumor overexpression of uPA, tPA and a mutant form of uPA, which does not bind to its receptor, but preserves the proteolytic activity.

Construction of mutant uPA clones and cell transfection. We chose to pursue a genetic approach, in which we stably transfected an aggressive and highly metastatic murine breast cancer cell line (4T1) with the genes for wild type uPA, tPA and a receptor binding mutant of uPA (uPAm). We inserted the cDNA encoding murine tPA into pcDNA 3.1 (+) (Invitrogen), a CMV driven mammalian expression vector, by restriction enzyme digestion of BamHI and NheI. Wild type uPA cDNA was inserted to the pcDNA 3.1 (+) expression vector and the transfected into 4T1 cells in a similar fashion as described for the tPA cDNA. Residues 22, 27, 29 and 30 on the growth factor domain of the murine uPA molecule have been implicated in binding to mouse uPAR.

Indeed, for mouse uPA, a triple mutation at residues 27, 29 and 30 has been shown to abrogate uPA binding to its receptor. We introduced these mutations by multi-site-directed mutagenesis using a clone of wild type UPA generated by RT-PCR of the murine cell line Lewis lung carcinoma RNA.

The oligonucleotides used to create the triple mutation changing Arg 27, 29, 30 into Asn 27, His 29, Trp 30 were as follows: (forward primer) 5′ P-CCT ACA AGT ACT TCT CCA ACA TTC ACT GGT GCA GCT GCC CAA GG 3′ and (reverse) 5′ P-CCT TGG GCA GCT GCA CCA GTG AAT GTT GGA GAA GTA CTT GTA GG 3′. The resulting mutant will have lost an EcoRI site at position 143, which was used to initially screen different mutant clones generated. After this, we inserted the mutant uPA cDNA into the pcDNA 3.1 (+) vector. Prior to introducing the vector constructs into the cancer cell lines, we sequenced the constructs and performed in vitro transcription-translation assays, in order to confirm that the DNA products were correct and that they transcribed.

The pcDNA 3.1-tPA, pcDNA 3.1-wild type uPA, pcDNA 3.1-uPAm constructs, as well as an empty vector control were introduced into 4T1 cells by transfection using lipofectamine (Gibco-BRL) reagent. We selected 12-14 stable single clones in each group with 500 ug/mL of hygromycin. Each of the clones was evaluated for generation of the gene of interest by northern blot. The clones that had the highest generation of the genes of interest were selected. These were: tPA clone 9, wild type uPA clone 11, and uPA mutant clone 11. Pools of stable clones of pcDNA 3.1-tPA and pcDNA 3.1 (+) were also selected using 500 ug/mL of hygromycin.

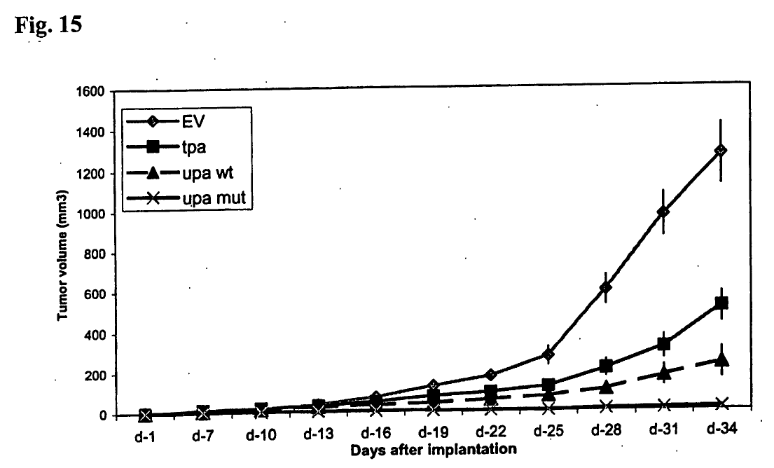

Experiments using transfected tumor cells. Tumor cells (10⁵ cells in 50 uL) were injected into the left 5th mammary pad of female (4-6) week old BALB/c mice (Charles River Labs, Cambridge, Mass.). Groups of 12 animals were injected with cells transfected with empty vector and cells transfected with tPA cDNA, wild type uPA cDNA, and mutant uPA cDNA. Primary tumor size was measured every other day for a total of five weeks using calipers and calculated using the standard formula (width2×length×0.52). FIG. 15 demonstrates the effects of tissue proteases in a murine breast cancer model. Results demonstrate primary tumor cells expressing tissue proteases are more resistant to tumor growth, relative to hygromycin-resistant 4T1 control cells, and that expression of mutants, defective for uPA receptor binding, is most effective in inhibiting tumor growth. Expression of wildtype uPA and tPA also significantly inhibit tumor cell growth, albeit less effectively. At 5 weeks post tumor cell implantation, animals were euthanized; and the primary tumors and lungs were removed and sectioned.

High throughput assays and screens. Other methods of observing changes to uPA-uPA-R interactions and subsequent biological activity may be exploited in high throughput assays for the purpose of identifying compounds that modulate this protein-protein interaction. Compounds that inhibit uPA from binding to uPA-R without affecting uPA proteolytic activity may be identified by such assays. Such identified compounds may have utility as therapeutic agents in the treatment of angiogenic disorders.

There are many methods known in the art to screen for modulators of protein-protein interactions and they are applicable here. One method is to immobilize one component (for example, the uPA-receptor) to a solid support matrix. The second component (for example, urokinase) is labeled (either through the use of radioactive isotopes such as ³²P or ³⁵S, or through non-radioactive alternatives such as fluorophores) and allowed to form a complex with the immobilized receptor. This complex is ready for screening with candidate compounds. A compound that displaces uPA from the uPA- receptor can be readily measured by release of radioactivity or fluorescence.

Alternatively, compounds can be contacted to immobilized uPA-R, followed by addition of the labeled uPA polypeptide. Compounds that modulate uPA-uPA-R interactions could also be measured by release of either radioactivity or fluorescence.

Therapeutic Uses

The invention features methods for treating angiogenesis associated diseases or disorders by administering polypeptide or nucleic acid compounds. Compounds of the present invention may be administered by any appropriate route for treatment or prevention of a disease or condition associated with angiogenesis associated diseases. These may be administered to humans, domestic pets, livestock, or other animals with a pharmaceutically acceptable diluent, carrier, or excipient, in unit dosage form. Administration may be parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, by suppositories, or oral administration.

Therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found, for example, in “Remington: The Science and Practice of Pharmacy” (20th ed., ed. A. R. Gennaro A R., 2000, Lippincott Williams & Wilkins). Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Nanoparticulate formulations (e.g., biodegradable nanoparticles, solid lipid nanoparticles, liposomes) may be used to control the biodistribution of the compounds. Other potentially useful parenteral delivery systems include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycholate and deoxycholate, or may be oily solutions for administration in the form of nasal drops, or as a gel. The concentration of the compound in the formulation will vary depending upon a number of factors, including the dosage of the drug to be administered, and the route of administration.

The compound may be optionally administered as a pharmaceutically acceptable salt, such as a non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acid such as hydrochloric acid, hydrobromic acid, sulfuric acid phosphoric acid, or the like. Metal complexes include zinc, iron, and the like.

Administration of compounds in controlled release formulations is useful where the compound of formula I has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD₅₀) to median effective dose (ED₅₀)); (ii) a narrow absorption window in the gastro-intestinal tract; or (iii) a short biological half-life, so that frequent dosing during a day is required in order to sustain the plasma level at a therapeutic level.

Many strategies can be pursued to obtain controlled release in which the rate of release outweighs the rate of metabolism of the therapeutic compound. For example, controlled release can be obtained by the appropriate selection of formulation parameters and ingredients, including, eg., appropriate controlled release compositions and coatings. Examples include single or multiple unit tablet or capsule compositions, oil solutions, suspensions, emulsions, microcapsules, microspheres, nanoparticles, patches, and liposomes.

Formulations for oral use include tablets containing the active ingredient(s) in a mixture with non-toxic pharmaceutically acceptable excipients. These excipients may be, for example, inert diluents or fillers (e.g., sucrose and sorbitol), lubricating agents, glidants, and antiadhesives (e.g., magnesium stearate, zinc stearate, stearic acid, silicas, hydrogenated vegetable oils, or talc).

Formulations for oral use may also be provided as chewable tablets, or as hard gelatin capsules wherein the active ingredient is mixed with an inert solid diluent, or as soft gelatin capsules wherein the active ingredient is mixed with water or an oil medium.

Administration of tissue proteases. By selectively disrupting or preventing tissue proteases, such as tPA and uPA, from binding to their natural receptor(s) the polypeptides of the invention, or derivatives or peptidomimetics thereof, can significantly decrease angiogenic potential resulting in reduction or ablation of neoplastic cell survival or growth. Therefore, the polypeptides of the invention, or derivatives or peptidomimetics thereof, can be used in the treatment of cancer or other neoplasms or even other angiogenesis-associated diseases (e.g., macular degeneration of the eye).

Angiogenesis-associated disorders include, cancer, rheumatoid arthritis, psoriasis, pyogenic granuloma, diabetic retinopathy, macular degeneration, corneal graft neovascularization, hypertrophic scarring, angiofibroma, Osler-Weber syndrome, neovascular glaucoma, and scleroderma.

Cancers and other neoplasms include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma).

For any of the methods of application described above, the tissue protease, fragment or mutant thereof, or peptidomimetic small molecule may be applied to the site of the needed therapeutic event (for example, by injection), or to tissue in the vicinity of the predicted therapeutic event or to a blood vessel supplying the cells predicted to require enhanced therapy.

The dosage of a tissue protease, fragment or mutant thereof, or peptidomimetic small molecule depends on a number of factors, including the size and health of the individual patient, but, generally, between 0.1 mg and 100 mg/kg body weight, is administered per day to an adult in any pharmaceutically acceptable formulation. In addition, treatment by any of the approaches described herein may be combined with more traditional therapies.

Combination therapy. If desired, polypeptides of the invention may be administered alone or in combination with a second, third, fourth, or even fifth therapeutic agent. Combination therapy may be performed alone or in conjunction with another therapy (e.g., surgery, y-radiation, chemotherapy, biologic therapy). Additionally, a person having a greater risk of developing a neoplasm (e.g., one who is genetically predisposed or one who previously had a neoplasm) may receive prophylactic treatment to inhibit or delay neoplastic formation. The duration of the combination therapy depends on the type of disease or disorder being treated, the age and condition of the patient, the stage and type of the patient's disease, and how the patient responds to the treatment.

The dosage, frequency and mode of administration of each component of the combination can be controlled independently. For example, one compound (i.e., the tissue protease) may be administered intravenously once per day, while the second compound (i.e., the antiproliferative) may be administered orally twice per day. Combination therapy may be given in on-and-off cycles that include rest periods so that the patient's body has a chance to recovery from any as yet unforeseen side-effects. The compounds may also be formulated together such that one administration delivers both compounds.

Exemplary antiproliferative agents, include alkylating agents (e.g., nitrogen mustards such as cyclophosphamide, ifosfamide, trofosfamide, and chlorambucil; nitrosoureas such as carmustine, and lomustine; alkylsulphonates such as bisulfan and treosulfan; triazenes such as dacarbazine; platinum-containing compounds such as cisplatin and carboplatin), plant alkaloids (e.g., vincristine, vinblastine, anhydrovinblastine, vindesine, vinorelbine, paclitaxel, and docetaxol), DNA topoisomerase inhibitors (e.g., etoposide, teniposide, topotecan, 9-aminocamptothecin (campto), irinotecan, and crisnatol), mytomycins (e.g., mytomicin C), antifolates (e.g., methotrexate, trimetrexate, mycophenolic acid, tiazofurin, ribavirin, EICAR, hydroxyurea, and deferoxamine), uracil analogs (5-fluorouracil, floxuridine, doxifluridine, and ratitrexed), cytosine analogs (cytarbine, cytosine arabinoside, and fludarabine), purine analogs (e.g., mercaptopurine, and thioguanine), hormonal therapies (e.g., tamoxifen, raloxifene, megestrol, goserelin, leuprolide acetate, flutamide, and bicalutamide), vitamin D3 analogs (EB 1089, CB 1093, and KH 1060), vertoporfin, phthalocyanine, photosensitizer Pc4, demethoxy-hypocrellin A, interferon-α, interferon-γ, tumor necrosis factor, lovastatin, 1-methyl-4-phenylpyridinium ion, staurosporine, actinomycin D, bleomycin A2, bleomycin B2, adriamycin, peplomycin, daunorubican, idarubican, epirubican, pirarubican, zorubican, mitoxantrone, verapamil, and the uPA octamer-capped peptide, A6.

Gene therapy. Gene therapy is another potential therapeutic approach in which nucleic acids encoding tissue proteases such as tPA and uPA, which are incapable of binding to their cognate receptor, are introduced into cells. The gene must be delivered to those cells in a form in which it can be taken up and encode for sufficient protein to provide effective function.

Transducing retroviral, adenoviral, and human immunodeficiency viral (HIV) vectors can be used for somatic cell gene therapy especially because of their high efficiency of infection and stable integration and expression (see, for example, Cayouette and Gravel, Hum. Gene Ther., 8:423-430, 1997; Kido et al. Curr. Eye Res., 15:833-844, 1996; Bloomer et al., J. Virol., 71:6641-6649, 1997; Naldini et al., Science 272:263-267, 1996; Miyoshi et al., Proc. Natl. Acad. Sci. USA, 94:10319-10323, 1997). For example, uPA nucleic acid, or portions thereof, can be cloned into a retroviral vector and driven from its endogenous promoter or from the retroviral long terminal repeat or from a promoter specific for the target cell type of interest (such as endothelial cells). Other viral vectors, which can be used, include adenovirus, adeno-associated virus, vaccinia virus, bovine papilloma virus, vesicular stomatitus virus, or a herpes virus such as Epstein-Barr Virus.

Gene transfer could also be achieved using non-viral means requiring infection in vitro. This would include calcium phosphate, DEAE-dextran, electroporation, and protoplast fusion. Liposomes may also be potentially beneficial for delivery of DNA into a cell. Although these methods are available, many of these are of lower efficiency.

Production of tissue protease containing vectors. Tissue proteases can be produced by any method known in the art for the expression of recombinant proteins. Nucleic acids that encode tissue proteases may be introduced into various cell types or cell-free systems for expression thereby allowing small-, large-, and commercial-scale production, purification, and patient therapy.

Eukaryotic and prokaryotic tissue protease expression systems may be generated in which a tissue protease-coding sequence is introduced into a plasmid or other vector, which is then used to transform living cells. Constructs in which the tissue protease cDNA contains the entire open reading frame or biologically active fragment thereof, are inserted in the correct orientation into an expression plasmid and may be used for protein expression. It is understood that the expression of tissue proteases in eukaryotic expression systems has the added benefit of being post-translationally processed in the appropriate cellular organelle(s). Secreted proteins can be processed by proteolytic processing by proteases residing at the extracellular face of the cell, such as the proprotein convertases (PCs). Optionally, production of tissue proteases, such as tPA and uPA can be attained by fusing the corresponding nucleic acid sequence immediately following an initiator methionine (AUG). Translation of the resulting mRNA in any prokaryotic or eukaryotic host would lead to the cleavage of the initiator methionine by a methionine aminopeptidase (MetAP). MetAPs have been extensively studied and have been shown to cleave the initiator methionine residue if the amino acid at position 2 (i.e., following the methionine) is glycine, alanine, serine, threonine, proline, cysteine, or valine (Arfine et al., Proc. Natl. Acad. Sci. USA, 92:7714-7718, 1995; Bradshaw et al., Trends Biochem. Sci., 23:263-267, 1998; Lowther and Matthews, Biochim. Biophys. Acta, 1477:157-167, 2000).

Tissue proteases can be expressed as soluble cytoplasmic proteins, or preferably, fused in-frame with a secretory signal peptide to be expressed as secreted recombinant polypeptides. Preferably, the secretory signal is based on either the A or α-factor secretory signal of Saccaromyces cereviseae. Prokaryotic and eukaryotic expression systems also allow for the expression and recovery of tissue protease fusion proteins in which the tissue protease is covalently linked to a tag molecule on either the amino terminal or carboxy terminal side, which facilitates identification and/or purification. Examples of tags that can be used include hexahistidine, HA, FLAG, and c-myc epitopes. Larger fusion tags may also be used and include glutathione-S-transferase, maltose binding protein, cellulose binding protein, and protein-A. An enzymatic or chemical cleavage site can be engineered between the tissue protease and the tag moiety so that the tag can be removed following purification.

If desired, the tissue protease may also be engineered as a fusion protein containing one member of a binding pair to facilitate protein purification. Exemplary binding pairs include without limitation antigen-antibody, biotin-avidin or biotin-strepavidin, hormone-hormone receptor, receptor-ligand, enzyme-substrate, IgG-protein A, and GST-glutathione.

Host Cells. Once a tissue protease expression vector is constructed, it is introduced into an appropriate host cell by transformation techniques, such as, but not limited to, calcium phosphate transfection, DEAE-dextran transfection, electroporation, bombardment, microinjection, protoplast fusion, dendrimer-mediated transfection, or liposome-mediated transfection. The host cells that are transfected with the vectors of this invention may include (but are not limited to) E. coli or other bacteria, yeast, fungi, insect cells (using, for example, baculoviral vectors for expression in Sf9 or Sf21 insect cells), or cells derived from murine, human, or other animals. Those skilled in the art of molecular biology will understand that a wide variety of expression systems and purification systems may be used to produce recombinant trefoil peptides and mixtures thereof. Suitable host cells include, for example, yeast, bacteria, insect cells, mammalian cells. Desirable yeast cells include Saccaromyces cereviseae, Schizosaccaromyces pombe, or the methylotrophic yeast, Pichia pastoris. Insect cells include Sf9 cells, Sf21, and Schneider cells. Mammalian cells include NIH-3T3, C3H10T1/2, HeLa, HEK293, COS, CV, and CHO cells.

Alternatively, bacterial host cells, such as E. coli may be used. In addition to E. coli, other bacterial species are also useful to propagate and/or express tissue proteases in a manner similar to using E. coli. For instance, Lactobacilli and Bifidobacterium species may be used to express the tissue protease either as soluble cytoplasmic proteins or by creating chimeric fusion proteins in which signal peptides would direct the expressed proteins into the periplasmic regions, to the outer surface of the bacteria, or as a secreted product out of the cell. Both Lactobacilli and Bifidobacterium spp can be further utilized to express foreign proteins in the preparation of consumable food products, for example, in making yogurt or other dairy products.

Protein Purification. Once a recombinant protein is expressed, it can be isolated from cell lysates if expressed as a cytoplasmic protein, or from the media if expressed as a secreted protein. Recombinant chimeric proteins bearing the A or α-factor secretory signal in yeast expression systems, for example, are exported out of the cell and can be collected from the culture media for further purification (see, for example, U.S. Pat. Nos. 4,808,537, 4,837,148, 4,879,231, 4,882,279, 4,818,700, 4,895,800, and 4,812,405, 5,032,516, 5,122,465, 5,268,273; hereby incorporated by reference).

Protein purification techniques such as ion-exchange, gel-filtration, and affinity chromatography can be utilized to isolate intestinal trefoil peptides from unwanted cellular proteins. Once isolated, the recombinant protein can, if desired, be purified further by high performance liquid chromatography (HPLC; e.g., see Fisher, Laboratory Techniques In Biochemistry And Molecular Biology, Work and Burdon, Eds., Elsevier, 1980).

If the tissue protease is fused in frame with a binding pair member, the 25 tissue protease can be isolated using a purification method based on the binding interaction. For example, a tissue protease fusion containing a biotin acceptor domain may be expressed in the yeast Pichia pastoris. Following in vitro biotinylation with biotin ligase and solubilization of crude membrane fractions with a detergent, the tissue protease can be purified using a combination of chromatographic techniques. Such a system is described in detail by Julien et al. (Biochemistry 39:75-85, 2000). Alternatively, if large-scale production of tissue proteases is required, any protein purification method known in the art may be used. An exemplary method is aqueous two phase systems and is described by Cunha T., et al (Mol. Biotechnol, 20: 29-40, 2002).

Protein modifications. It is desirable that tissue proteases of the invention lack receptor binding activity. For example, it is known that amino acids 24-30 (the Ω-loop) of uPA is important for receptor association. Substitutions at any of these residues, or residues residing outside this loop and identified to regulate binding of uPA to its receptor, would be of benefit. In a desirable embodiment, amino acid substitutions are directed to the sequence ²⁴tyr-²⁵phe-²⁶ser-²⁷asn-²⁸ile-²⁹his-³⁰trp in human, ²⁴tyr-²⁵phe-²⁶ser-²⁷arg-²⁸ile-²⁹arg-³⁰arg in mouse, and ²⁴tyr-²⁵phe-²⁶ser-²⁷ser-²⁸ile-²⁹arg-³⁰arg in rat. In another desirable embodiment, any 2, 3, 4, 5, 6, or all 7 amino acids of uPA may be substituted with another amino acid, typically a non-conservative amino acid. Amino acid residues in the Ω-loop may be substituted from one species to another. For example, a triple mutant of murine uPA incorporating the human amino acid residue substitutions at positions 27, 29, and 30 (i.e., R27N, R29H, and R30W) has been shown to ablate binding of murine uPA to the mouse UPA-R receptor.

Receptor binding mutants of uPA may further possess mutations or modifications modulating biological activity, for example, increased, decreased, or ablated catalytic activity, mutations affecting protein phosphorylation (e.g., Ser138), and mutations affecting substrate specificity.

Advanced protein engineering technologies can be incorporated to develop human protein pharmaceuticals with enhanced therapeutic properties. Most protein pharmaceuticals are rapidly eliminated by the body, which limits their effectiveness and requires that they be administered by frequent, often daily, injection. The most commonly employed method for extending protein half-life is PEGylation. PEGylating proteins uses compounds such as N-hydroxysuccinimide (NHS)-PEG to attach PEG to free amines, typically at lysine residues or at the N-terminal amino acid. The PEG moiety attaches to the protein randomly at any of the available free amines, resulting in a heterogeneous product mixture consisting of mono-, di-, tri-, etc., PEGylated species modified at different lysine residues.

Site-specific PEGylation may be employed. Site-Specific PEGylation allows a protein to be selectively modified with PEG at a single, unique, pre-determined site. The site of PEGylation potentially can be any amino acid position in the protein and can be varied depending upon the protein. By targeting the PEG molecule to an optimal site in a protein, it is possible to create PEGylated proteins that are homogeneously modified and have no significant loss of biological activity.

An alternative technology takes advantage of the modular structure and long circulating half-lives of human immunoglobulins (antibodies). Recombinant DNA methods are employed to covalently fuse therapeutic proteins to the Fc domains of human immunoglobulin gamma proteins (IgGs). IgGs are abundant proteins that have circulating half-lives of up to 21 days in humans. In a desirable embodiment, human or humanized immunoglobins are used as a fusion. It is contemplated that the immunoglobins may also be PEGylated and glycosylated to further increase half-life, or decrease immune detection.

Fusion tags or post-translational modifications can be used with the tissue proteases. For example, the tissue proteases can be asialo-glycosylated. It is known that liver cells express the asialo-glycoprotein receptor. Asialo-glycosylated proteins would thus accumulate in the liver, increasing therapeutic bioavailability in pathologies affecting the liver. Analogous fusion tags and/or modifications can be incorporated to the polypeptides of the invention directing them to specific organs or tissues. For example, fusion tags can be made with non-functional EGF-Rc-binding epidermal growth factor; many breast cancers have been found to over-express the EGF receptor and a non-functional epidermal growth factor would be able to target the tissue proteases to the cancerous cells.

Test extracts and compounds. In general, compounds that affect PAR or uPA/uPA-R receptor signaling are identified from large libraries of both natural products, synthetic (or semi-synthetic) extracts or chemical libraries, according to methods known in the art.

Those skilled in the art will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modifications of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from, for example, Brandon Associates (Merrimack, N.H.) and Aldrich Chemical (Milwaukee, Wis.).

Alternatively, libraries of natural compounds in the form of bacterial, fungal, plant, and animal extracts are commercially available from a number of sources, including, but not limited to, Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art (e.g., by combinatorial chemistry methods or standard extraction and fractionation methods). Furthermore, if desired, any library or compound may be readily modified using standard chemical, physical, or biochemical methods.

Other Embodiments. From the foregoing description, it is apparent that variations and modifications may be made to the invention described herein to adopt it to various usages and conditions. Such embodiments are also within the scope of the following claims.

All publications mentioned in this specification are herein incorporated by reference to the same extent as if each independent publication or patent application was specifically and individually indicated to be incorporated by reference.

1. A method for the treatment of angiogenesis-associated diseases, said method comprising administering a therapeutic amount of a pharmaceutical composition comprising a Protease-Activated Receptor (PAR) agonist, wherein said agonist is capable of binding directly to the PAR receptor.

2. A method for the treatment of angiogenesis-associated diseases, said method comprising administering a therapeutic amount of a compound which results in activation of a Protease-Activated Receptor (PAR), wherein said treatment does not comprise administering either tissue plasminogen activator (tPA) polypeptide or a urokinase plasminogen activator (uPA), wherein said uPA is capable of binding to the human uPA receptor (uPA-R) in combination with captopril.

3. A pharmaceutical composition comprising (i) substantially pure PAR-agonist, wherein said agonist is capable of binding directly to the PAR receptor; and (ii) a pharmaceutically acceptable carrier.

4. A pharmaceutical composition comprising (i) a therapeutic amount of a compound which results in activation of PAR receptor, wherein said composition does not comprise either tPA polypeptide or uPA, wherein said uPA is capable of binding to the human uPA receptor; and (ii) a pharmaceutically acceptable carrier.

5. A method for the treatment of angiogenesis-associated diseases, said method comprising administering a therapeutic amount of a pharmaceutical composition comprising thrombin or prothrombin to a patient diagnosed with an angiogenesis associated disease.

6. A method for the treatment of angiogenesis-associated diseases, said method comprising administering a pharmaceutical composition comprising a compound that modulates PAR biological activity, wherein said treatment does not comprise administering either tPA polypeptide or a uPA, wherein said uPA is capable of binding to the human uPA-R if said treatment also comprises administering captopril.

7. A method for identifying candidate compounds that modulate PAR biological activity, said method comprising the steps of:
a. contacting said Protease-Activated Receptor to a candidate compound; and
b. measuring binding of said compound to said PAR receptor,
wherein said binding identifies said candidate compound as a compound that is useful for modulating PAR biological activity.

8. A method for the treatment of angiogenesis-associated diseases, said method comprising administering a pharmaceutical composition comprising substantially pure urokinase (uPA) polypeptide, wherein said polypeptide is incapable of binding to the urokinase receptor, uPA-R.

9. A method for the treatment of angiogenesis-associated diseases, said method comprising introducing a transgene encoding a uPA polypeptide, wherein said uPA polypeptide is incapable of binding to uPA-R, to a cell, said transgene is operably linked to expression control sequences, and said transgene being positioned for expression in said cell.

10. A method for the treatment of angiogenesis-associated diseases, said method comprising introducing a transgene encoding a PAR polypeptide, said transgene is operably linked to expression control sequences, and said transgene being positioned for expression in said cell.

11. A method for identifying antiangiogenic molecules in serum plasma, said method comprising:
a. contacting said serum plasma with a tissue protease and an ACE inhibitor;
b. depleting said plasma of angiostatin;
c. chromatographically separating plasma fractions; and
d. determining angiogenic potential of said fraction,
wherein, inhibition of angiogenesis identifies said fraction as antiangiogenic.

12. The method of claims 1, 2, 5,6, or 8-10, wherein said angiogenesis-associated diseases is selected from the group consisting of cancer, rheumatoid arthritis, psoriasis, pyogenic granuloma, HIV Kaposi's sarcoma, diabetic retinopathy, macular degeneration, corneal graft neovascularization, and hypertrophic scarring.

13. The method of claim 12, wherein said angiogenesis-associated disease is cancer.

14. The method of claims 1-4, 6, 7, or 10, wherein said Protease-Activated Receptor is selected from the group consisting of PAR-1, PAR-3, and PAR-4.

15. The method of claims 1-4, or 6, wherein said PAR-agonist or activator of the PAR receptor is selected from the group consisting of the polypeptides, SFLLRNPNDKYEPF, SFLLRN, SALLRN, GYPGKF, and SLIGKV.

16. The method of claims 1-4, or 6, wherein said PAR-agonist is a monoclonal antibody.

17. The method of claim 16, wherein said monoclonal antibody modulates PAR-receptor signaling.

18. The method of claims 16 or 17, wherein said monoclonal antibody further prevents receptor internalization.

19. The method of claim 5, wherein said treatment further comprises administering an anti-coagulant.

20. The method of claims 1, 2, 5, 6, or 8-10, wherein said treatment further comprises administering an ACE inhibitor.

21. The method of claim 20, wherein said ACE inhibitor is selected from a group consisting of: captopril, enalapril, lisinopril, benazepril, fosinopril, ramipril, quinapril, perindopril, trandolapril, and moexipril.

22. The method of claim 11, wherein said serum plasma is mammalian serum plasma.

23. The method of claim 11, wherein said tissue protease is selected from a group consisting of urokinase, tissue plasminogen activator, and streptokinase.

24. The method of claim 11, wherein said fraction having antiangiogenic activity is further purified to allow for identification.

25. The method of claim 8 or 9, wherein said uPA is mammalian.

26. The method of claim 25, wherein said uPA is mouse, rat, or human.

27. The method of claim 26, wherein said uPA is human uPA.

28. The method of claim 26, wherein said human uPA further comprises amino acid substitutions within the Ω-loop.

29. The method of claim 28, wherein said Ω-loop comprises amino acid residue substitutions on the amino acid sequences of the group consisting of the sequence ²⁴tyr-²⁵phe-²⁶ser-²⁷asn-²⁸ile-²⁹his-³⁰trp in human, ²⁴tyr-²⁵phe-²⁶ser-²⁷arg-²⁸ile-²⁹arg-³⁰arg in mouse, and ²⁴tyr-²⁵phe-²⁶ser-²⁷ser-²⁵ile-²⁹arg-³⁰arg in rat.

30. A pharmaceutical composition comprising (i) a therapeutic amount of a uPA, wherein said uPA is incapable of binding to the uPA-receptor; and (ii) a pharmaceutically acceptable carrier.

31. The pharmaceutical composition of claim 30, wherein said uPA comprises amino acid substitutions within the Ω-loop.

32. The pharmaceutical composition of claim 31, wherein said uPA is mouse, rat, or human uPA..

33. The pharmaceutical composition of claim 32, wherein said uPA is human uPA.

34. The pharmaceutical composition of claim 32, wherein said uPA comprises any three amino acid residue substitutions of the sequence ²⁴tyr-²⁵phe-³⁶ser-²⁷asn-²⁸ile-²⁹his-³⁰trp in human, ²⁴tyr-²⁵phe-²⁶ser-²⁷arg-²⁸ile-²⁹arg-³⁰arg in mouse, and ²⁴tyr-²⁵phe-²⁶ser-²⁷ser-²⁸ile²⁹arg-³⁰arg in rat.

35. The pharmaceutical composition of any of claims 30-34, wherein said pharmaceutical composition is used for the treatment of an angiogenesis-associated disease.

36. The pharmaceutical composition of claim 35, wherein said an angiogenesis-associated disease is cancer.

37. The pharmaceutical composition of claim 36, wherein said cancer is breast cancer.

38. The pharmaceutical composition of any of claims 30-37, wherein said composition further comprises a second therapeutic agent.

39. The pharmaceutical composition of claim 38, wherein said second therapeutic agent is an antiproliferative agent.

40. The method of claim 8 or 9, wherein said method further comprises administering a therapeutic amount of an antiproliferative agent simultaneously or within 14 days of each other in amounts sufficient to inhibit the growth of said neoplasm.

41. The method of claim 8 or 9, wherein said method further comprises administering a therapeutic amount of an antiproliferative agent.

42. The method of claim 9 or 10, wherein said transgene is operably linked to tissue-specific expression control sequences.
